(19) United States
(12) Patent Application Publication
(10) Pub. No.: US 2005/0220774 A1
(43) Pub. Date: Oct. 6, 2005

(54) Methods of inducing differentiation in ex vivo expanded stem cells

(76) Inventors: Tony Peled, Mevaseret Zion (IL); Avi Troves, Mevaseret Zion (IL)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/508,244

(22) PCT Filed: Mar. 18, 2003
(86) PCT No.: PCT/IL03/00235

§ 371 Date: May 19, 2005

Related U.S. Application Data
(60) Provisional application No. 60/364,590, filed on Mar. 18, 2002.
(60) Provisional application No. 60/452,545, filed on Mar. 7, 2003.

(30) Foreign Application Priority Data
Jan. 23, 2003 (WO) PCT/IL03/00062
Jan. 26, 2003 (WO) PCT/IL03/00064

(51) Int. Cl. A61K 48/00

(52) U.S. Cl. 424/93.21; 424/93.7

(57) ABSTRACT

Method of differentiating ex vivo expanded stem cells in-tissue anti in vivo are provided. Also provided are method of treating individuals suffering from a disorder necessitating cell replacement or tissue replacement therapy using ex vivo expanded stem cells.

### FIELD AND BACKGROUND OF THE INVENTION

The present invention relates to methods of inducing differentiation in ex vivo expanded stem cells, and more particularly, in one embodiment, to the use of ex vivo expanded hematopoietic stem cells for the re-generation of damaged tissues such as heart and lung and thus the use of such cells in treatment of a variety of disorders.

Stem Cells and Their Therapeutic Potential

Stem cells are primitive cells having the capacity to mature into other cell types, for example, brain, muscle, liver and blood cells. Stem cells are typically classified as either embryonic stem cells, or adult tissue derived-stem cells, depending on the source of the tissue from which they are derived.

Pluripotent human stem cells provide biomedical research with new approaches for drug development and testing and for organ repair and replacement.

Unlike all current treatments relying upon surgical intervention or drugs that modulate physiological activities, stem cells provide a replacement for dysfunctional or degenerating tissue. Using stem cells, replacement therapy could dramatically change the prognosis of many untreatable diseases.

For example, many neurological diseases, such as disorders of the brain, spinal cord, peripheral nerves and muscles, are characterized by the sudden or gradual death of brain or muscle cells. These diseases which include stroke, head and spinal cord trauma, Alzheimer's Disease, Parkinson's Disease, multiple sclerosis, amyotrophic lateral sclerosis (ALS), genetic enzyme deficiencies, muscular dystrophy and others could be potentially treated using stem cell replacement therapy.

The recent discoveries that hematopoietic stem cells can give rise to non-hematopoietic tissues suggest that these cells may have greater differentiation potential than was previously assumed and open new frontiers for their therapeutic applications [Petersen, B. E. et al. Bone marrow as a potential source of hepatic oval cells. Science 284, 1168-1170 (1999); Brazelton, T. R. et al. (2000). From marrow to brain: expression of neuronal phenotypes in adult mice. Science 290, 1775-1779; Mezey, E., et al. (2000). Turning blood into brain: cells bearing neuronal antigens generated in vivo from bone marrow. Science 290, 1779-1782; Lagasse, E. et al. (2000). Purified hematopoietic stem cells can differentiate to hepatocytes in vivo. Nature Med. 6, 1229-1234; Krause, D. S. et al. (2001). Multi-organ, multi-lineage engraftment by a single bone marrow derived stem cell. Cell 105, 369-377].

Studies have shown that cord blood-derived stem cells are capable of repairing neurological damage caused by brain injuries and strokes [Lu D et al. (2002). Intravenous administration of human umbilical cord blood reduces neurological deficit in the rat after traumatic brain injury. Cell Transplant. 11:275-81] and are also capable of functional and morphological incorporation into animal heart tissue [Orlic, D. et al., Mobilized bone marrow cells repair the infarcted heart, improving function and survival (2001). Proc. Natl. Acad. Sci. USA 98: 10344-9; Orlic, D. et al., Transplanted adult bone marrow cells repair myocardial infarcts in mice (2001). Ann N Y Acad. Sci. 938: 221-9, discussion 229-30; Orlic, D. et al., Bone marrow cells regenerate infarcted myocardium (2001). Nature, 410: 701-5].

Hematopoietic Stem Cells

Normal production of blood cells (hematopoiesis) and other cell types involves processes of proliferation and differentiation. In most hematopoietic cells, following division, the daughter cells undergo a series of progressive changes which culminate in fully differentiated (mature) functional blood cells which are mostly devoid of proliferative potential. Thus, the process of differentiation limits and eventually halts cell division. In only a small sub-population of hematopoietic cells, known as stem cells, can cell division result in progeny which are similar or identical to their parental cells. This type of cell division, known as self-renewal, is an inherent property of stem cells and helps to maintain a small pool of stem cells in their most undifferentiated state. Some stem cells lose their self-renewal capacity and following cell division differentiate into various types of lineage-committed progenitors which finally give rise to mature cells. While the latter provide the functional capacity of the blood cell system, the remaining stem cells are responsible for maintaining hematopoiesis throughout life despite a continuous loss of the more differentiated cells through apoptosis (programmed cell death) and/or active removal of aging mature cells by the reticulo-endothelial system.

As is discussed hereinabove, self-renewal of hematopoietic stem and progenitor cells, both in vivo and in vitro, is limited by cell differentiation. Differentiation in the hematopoietic system involves, among other changes, altered expression of surface antigens [Sieff C, Bicknell D, Caine G, Robinson J, Lam G, Greaves M F (1982) Changes in cell surface antigen expression during hematopoietic differentiation. Blood 60:703]. In normal tissue, most of the hematopoietic pluripotent stem cells and the lineage committed progenitor cells are CD34+. The majority of cells are CD34+CD38+, with a minority of cells (<10%) being CD34+CD38−. The CD34+CD38− phenotype characterizes the most immature hematopoietic cells which are capable of self-renewal and multi-lineage differentiation. A CD34+CD38− cell fraction contains more long-term culture initiating cells (LTC-IC) pre-CFU and exhibits longer maintenance of their phenotype and delayed proliferative response to cytokines as compared with CD34+CD38+ cells. Cells displaying the CD34+CD38− phenotype can give rise to lymphoid and myeloid cells in vitro and have an enhanced capacity to repopulate immune-deficient mice [Bhatia M, Wang J C Y, Kapp U, Bonnet D, Dick J E (1997) Purification of primitive human hematopoietic cells capable of repopulating immune-deficient mice. Proc Natl Acad Sci USA 94:5320]. Moreover, in patients receiving autologous blood cell transplantation, the number of CD34+CD38− cells infused correlates positively with the speed of hematopoietic recovery. Consistent with their function, CD34+CD38− cells have been shown to have detectable levels of telomerase, an enzyme associated with cell proliferation and prevention of DNA damage leading to apoptosis.

Ex Vivo Expansion of Hematopoietic Stem Cells

Despite their significant therapeutic potential, hematopoietic stem cells are not widely used in cell replacement and tissue regeneration therapies. This is partially due to their low availability and their limited capacity for expansion in common ex vivo culturing methods.

Ex Vivo Expansion in the Presence of Cytokines

A variety of protocols have been suggested and tested for enrichment of stem cell populations. The main experimental strategies employed include incubation of mononuclear cells with or without selection of CD34+ (Sandstrom C E, et al. Effects of CD34+ cell selection and perfusion on ex vivo expansion of peripheral blood mononuclear cells. Blood, 86: 958, 1995); with different cocktails of early and late growth factors [Petzer A L, Zandstra P W, Piret J M, Eaves C J. Differential cytokine effects on primitive (CD34+CD38−) human hematopoietic cells: novel responses to FLT3-ligand and thrombopoietin. J Exp Med 183: 2551, 1996]; with or without serum (Lebkowski J S, et al. Rapid isolation and serum-free expansion of human CD34+ cells. Blood Cells 20:404, 1994); in stationary cultures, rapid medium exchanged cultures (Schwartz R M, et al. In-vitro myelopoiesis stimulated by rapid medium exchange and supplementation with hematopoietic growth factors. Blood 78:3155, 1991) or under continuous perfusion (bioreactors) (Koller M R, Emerson S G, Palsson B O. Large-scale expansion of human stem and progenitor cells from bone marrow mononuclear cells in continuous perfusion cultures. Blood, 82: 378, 1993); and with or without established stromal cell layer (Verfaillie C M. Can human hematopoietic stem cells be cultured in vivo? Stem Cells 12: 466, 1994).

Although a significant expansion of intermediate and late progenitors was often obtained during 7-14 days ex vivo cultures, the population of early hematopoietic (CD34+CD38−) stem cells with high proliferative potential, usually declined [Koller M R, Emerson S G, Palsson B O. Large-scale expansion of human stem and progenitor cells from bone marrow mononuclear cells in continuous perfusion cultures. (1993). Blood, 82: 378; Haylock D N, et al. Ex vivo expansion and maturation of peripheral blood CD34+ cells into the myeloid lineage. (1992). Blood, 80: 1405; Brugger W, et al. Ex vivo expansion of enriched peripheral blood CD34+ progenitor cells by stem cell factor, interleukin-1 beta (IL-1 beta), IL-6, IL-3, interferon-gamma, and erythropoietin. (1993). Blood, 81: 2579; Sato N, et al. In vitro expansion of human peripheral blood CD34+ cells. (1993). Blood, 82: 3600].

Since cell replacement therapy and tissue regeneration require large amounts of stem cells, improved ex vivo expansion methods have been recently developed for stem cells.

In order to achieve maximal ex vivo expansion of stem cells, it is important that differentiation be reversibly inhibited or delayed, and that self-renewal of stem cells be maximally prolonged.

Stem Cells Expansion in the Presence of Transitional Metal Chelators

The use of copper chelators for ex vivo expansion of stem cells is based on the association between copper deficiency and hematological abnormalities such as anemia, neutropenia and thrombocytopenia.

The mechanism by which copper deficiency leads to neutropenia is unknown. Among the possible causes, either alone or in combination, are: (i) early death of progenitor cells in the BM; (ii) impaired formation of neutrophils from progenitor cells in the BM; (iii) decrease in cellular maturation rate in the BM; (iv) impaired release of neutrophils from the BM to the circulation; (v) enhanced elimination rate of circulating neutrophils.

Examination of the BM of neutropenic copper-deficient patients demonstrates the absence of mature cells (“maturation arrest”). It has been shown that cells derived from such BM did not form colonies in semi-solid medium containing copper-deficient serum, but retained the potential for normal colony growth in copper-containing serum. These results indicate the presence of intact progenitors in the patient's BM, and suggest that the block in development occurs after completion of the progenitor stage [Zidar B L et al. Observation on the anemia and neutropenia of human copper deficiency. (1977). Am. J. Hematol. 3: 177; Hirase N, et al. Anemia and neutropenia in a case of copper deficiency: Role of copper in normal hematopoiesis. (1992). Acta. Haematol. 87: 195].

International Patent Applications Serial Nos. PCT/IL99/00444 and PCT/US99/02664, U.S. patent application Ser. Nos. 09/986,897 09/988,127, and Peled et al. (Brit. J. Haematol. 116:655, 2002) teach that certain trace-element chelators, copper chelators in particular, can inhibit differentiation of stem and progenitor cells, thereby prolonging cell proliferation and expansion ex vivo. It is further disclosed that elevation of cellular copper content accelerates stem or progenitor cells differentiation. It was thus proposed that cellular copper is involved in the modulation of stem or progenitor cell self-renewal, proliferation and differentiation: increasing cellular copper content accelerates differentiation of stem or progenitor cells, while decreasing of cellular copper content inhibits differentiation of stem or progenitor cells. Indeed, ex vivo expansion of CD34+ cells in the presence of the copper chelator, tetra-ethylepentamine, TEPA, and high or low doses of early-acting cytokines [e.g., stem cell factor (SCF), FLT3, interleukin-6 ([L-6), thrombopoietin (TPO)], or a combination of early and late acting cytokines (e.g., G-CSF and GM-CSF) resulted in significant increases of cell clonability and percentage of CD34+ cells (U.S. patent application Ser. No. 09/986,897). Moreover, the addition of TEPA to long-term cultures (3-5 weeks) resulted in a more efficient clonability of cultures supplemented with either early cytokines or with both early and late cytokines (U.S. patent application Ser. No. 09/986,897). Similar effects were observed with other transition metal chelators such as captopril (CAP) or penicilamine (PEN), and other polyamines such as EDA, PEHA and TETA.

In addition, when TEPA-treated cultures were supplemented with copper, TEPA activities were reversed. However, when TEPA treated cultures were supplemented with other metal ions such as iron and selenium, TEPA effects were not reversed. In addition, when cell were exposed to zinc, which is known to interfere with transition metal metabolism, along with TEPA, the effects of TEPA on stem cell expansion and clonability was even more pronounced (U.S. patent Ser. No. 09/986,897).

Stem Cells Expansion Using Retinoic Acid Receptor Antagonists and Nicotinamide

As is described hereinabove, most of the hematopoietic pluripotent stem cells which are capable of self-renewal and multi-lineage differentiation are CD34+CD38−.

CD38 is a member of an emerging family of cytosolic and membrane-bound enzymes whose substrate is nicotinamide adenine dinucleotide (NAD), a coenzyme ubiquitously distributed in nature. In humans, CD38 is a 45 kDa type II trans-membrane glycoprotein. Recently, it has been demonstrated that CD38 is a multifunctional enzyme that exerts both NAD⁺ glycohydrolase activity and ADP-ribosyl cyclase activity and is thus able to catalyze the production of nicotinamide, ADP-ribose (ADPR), cyclic-ADPR (cADPR) and nicotinic acid adenine dinucleotide phosphate (NAADP) from its substrates [Howard et al. (1993) Science 252:1056-1059; Lee et al. (1999) Biol. Chem. 380:785-793]. The soluble domain of human CD38 catalyzes the conversion of NAD⁺ to cyclic ADP-ribose and to ADP-ribose via a common covalent intermediate [Sauve, A. et al. (2000) J. Am. Chem. Soc. 122: 7855-7859].

Experiments utilizing several leukemia cell lines revealed that retinoic acid receptor (RAR)-mediated signaling results in the induction of expression of the differentiation marker CD38 cell surface antigen, whereas antagonists to RAR abolished CD38 antigen up-regulation [Kapil M., et al. Involvement of retinoic acid receptor mediated signaling pathway in induction of CD38 cell surface antigen, Blood. (1997). 89: 3607-3614; Ueno H, et al. A novel retinoic acid receptor (RAR)-selective antagonist inhibits differentiation and apoptosis of HL-60 cells: implications of RAR alpha-mediated signals in myeloid leukemic cells. Leuk Res. (1998). 22: 517-25]. In addition, inhibition of CD38 by the CD38 inhibitor, nicotinamide, or by targeting CD38 mRNA using antisense oligonucleotides was found to affect the cADPR signal transduction pathway and inhibit differentiation [Munshi C B, et al. (2002). J. Biol. Chem. 277: 49453-8].

Nicotinamide (NA) is a water-soluble derivative of vitamin B, whose physiological active forms are nicotinamide adenine dinucleotide (NAD+/NADH) and nicotinamide adenine dinucleotide phosphate (NADP+/NADPH). The physiological active forms of NA serve as coenzyme in a variety of important metabolic reactions.

Retinoic acid (RA), the natural acidic derivative of Vitamin A (retinol) is an important regulator of embryonic development and it also influences the growth and differentiation of a wide variety of adult cell types. The biological effects of RA are generally mediated through their interaction with specific ligand-activated nuclear transcription factors, their cognate RA receptors (RARs). Receptors of the retinoic acid family comprise RARs, RXRs, Vitamin D receptors (VDRs), thyroid hormone receptors (THRs) and others. When activated by specific ligands these receptors behave as transcription factors, controlling gene expression during embryonic and adult development.

As disclosed in PCT/IL03/00064, nicotinamide, the CD38 inhibitor, represses the process of differentiation of stem cells and stimulates and prolongs the phase of active cell proliferation and expansion ex vivo. In addition, a series of chemical agents such as antagonists of the RAR, RXR and VDR also repress the process of differentiation of stem cells and stimulates and prolongs, for up to 16-18 weeks, the phase of active cell proliferation and expansion ex vivo.

As is further disclosed in PCT/IL03/00064 primary hepatocyte cultures incubated with agents such as retinoic acid receptor antagonists of the RAR and RXR super families, revealed an increase in the proportion of cells producing α-fetoprotein, indicating induction of proliferation of early hepatocyte populations. Antagonist-treated hepatocyte cultures grown without cytokines persisted for at least 3 weeks in culture, a finding which is in stark contrast to previous data which illustrated that growing primary hepatocytes for extended periods of time in culture was practically impossible, especially in the absence of cytokines [Wick M, et al. ALTEX. (1997). 14: 51-56; Hino H, et al. Biochem Biophys Res Commun. (1999). 256: 184-91; Tateno C, and Yoshizato K. Am J Pathol. (1996). 148: 383-92]. Supplementation with growth factors alone was insufficient to stimulate hepatocyte proliferation, and only RAR antagonist treatment of hepatocyte cultures resulted in the proliferation of early hepatocyte populations and in their persistence in culture, evident even following first and second passages.

Therefore, according to the teachings of PCT/IL03/00064, ex vivo expansion of stem cells of hematopoietic and other origins, can be achieved using molecules which interfere with CD38 expression and/or activity and thereby induce ex vivo and/or in vivo expansion of stem cell populations. Expansion of stem cell populations by this method can be used, for example, with hematopoietic stem cells to produce large numbers of undifferentiated CD34⁺/Lin⁻ (CD33, CD14, CD15, CD4, etc.), as well as CD34⁺/CD38⁻ cells, especially CD34⁺_{dim}/Lin⁻ cells.

Stein Cell Expansion in the Presence of PI-3 Kinase Inhibiting Agents

Phosphatidylinositol 3-kinase (PI 3-kinase) is a lipid kinase composed of a Src homology 2 domain-containing regulatory subunit (p85) and a 110-kD catalytic subunit (p110). PI 3-kinase catalyzes the formation of inositol phospholipids phosphorylated at the D3 position of PIPI 3-kinase. It was shown that the PI 3-kinase inhibitors wortmannin and LY294002 prevent increased CD38 mRNA expression and the over-expression of membrane CD38 antigen, as well as preventing expression of CD157, a CD38-related antigen on HL-60 and normal marrow CD34+ cells exposed to retinoic acid [Lewandowski, D., et al. (2002). Phosphatidylinositol 3-kinases are involved in the all-trans retinoic acid-induced upregulation of CD38 antigen on human hematopoietic cells. Br. J. Haematol. 118: 535-44].

Downstream signal transduction imposed by nuclear receptors such as the RARs, RXRs, VDRs and THRs may also be inhibited by inhibition of PI 3-kinase, which is an obligatory factor for proper receptor signaling. The critical function of PI 3-kinase in the activation of nuclear receptors such as VDR was demonstrated in THP-1 cells. Treatment of THP-1 cells with 1α,25-dihydroxyvitamin D₃ (D₃) was associated with rapid and transient increases in PI 3-kinase activity, as well as, with maturation of myeloid cells and surface expressions of CD 14 and CD11b, markers of cell differentiation. Induction of CD14 and CD11b expression in response to D₃ was reversed by (a) the PI 3-kinase inhibitors LY294002 and wortmannin; (b) antisense oligonucleotides to mRNA for the p110 catalytic subunit of PI 3-kinase; and (c) a dominant negative mutant of PI 3-kinase. Similarly, LY294002 and wortmannin inhibited D₃-induced expression of both CD14 and CD11b in peripheral blood monocytes. Western blots and in vitro kinase assays performed on immunoprecipitates of the VDR showed that D₃ treatment brought about formation of a complex containing both PI 3-kinase and the VDR. These findings reveal a novel, nongenomic mechanism of hormone action regulating monocyte differentiation, in which vitamin D₃ activates a VDR and PI 3-kinase-dependent signaling pathway [Hmama, Z., et al. (1999). 1alpha,25-dihydroxyvitamin D₃-induced myeloid cell differentiation is regulated by a vitamin D receptor-phosphatidylinositol 3-kinase signaling complex. J. Exp. Med. 190:1583-1594].

The functionality of PI 3-kinase as an obligatory downstream factor in the cellular pathways involved in induction of leukemic cell differentiation was also demonstrated in HL-60 cells that were induced to granulocytic differentiation by all-trans-retinoic acid. Immunochemical and immunocytochemical analyses by confocal microscopy revealed an increase in the amount of PI 3-kinase, which is particularly evident at the nuclear level. Inhibition of PI 3-kinase activity by nanomolar concentrations of wortmannin and of its expression by transfection with an antisense fragment of p85α prevented the differentiation process. The data obtained indicate that PI 3-kinase activity plays an essential role in promoting granulocytic differentiation [Bertagnolo, V. et al. (1999). Phosphoinositide 3-kinase activity is essential for all-trans-retinoic acid-induced granulocytic differentiation of HL-60 cells. Cancer Research 59: 542-546].

The involvement of PI 3-kinase in cell differentiation regulatory pathways was demonstrated also in non-hematopoietic cells. Smooth Muscle Cells (SMC) de-differentiation is induced by platelet-derived growth factor-BB (PDGF-BB), basic fibroblast growth factor (bFGF) and epidermal growth factor (EGF), even in the presence of insulin-like growth factor I (IGF-I) (inducer of a differentiated phenotype) in culture. This demonstrated that distinctly different signaling pathways regulate the SMC phenotype. Both the ERK and p38MAPK pathways triggered by PDGF-BB, bFGF, and EGF were found to play an essential role in inducing SMC de-differentiation, whereas the PI 3-kinase/protein kinase B (Akt) pathway was critical in maintaining a differentiated state. The same signaling pathways involving in the phenotypic determination of gizzard SMCs were observed in vascular SMCs. Thus, changes in the balance between the PI 3-kinase/protein kinase B (Akt) pathway and the ERK and p38MAPK pathways could determine phenotypes of visceral and vascular SMCs. [Ken'ichiro Hayashi, et al. Changes in the balance of phosphoinositide 3-kinase/protein kinase B (Akt) and the mitogen-activated protein kinases (ERK/p38MAPK). Determine a phenotype of visceral and vascular smooth muscle cells. J. Cell Biol. (1999). Volume 145: 727-740].

Therefore, several differentiation-inducing agents can activate PI 3-kinase, and the inhibition of the PI 3K/p70S6K pathway blocks the process of differentiation in these cell lines [Marcinkowska E (1999). Does the universal “signal transduction pathway of differentiation” exist? Comparison of different cell differentiation experimental models with differentiation of HL-60 cells in response to 1,25-dihydroxyvitamin D3. Postepy. Hig. Med. Dosw. 53: 305-13].

In addition, it was reported that cellular PI 3-kinase activity was strongly enhanced after exposure to Cu⁺⁺ [Ostrakhovitch E A et al. (2002). Copper ions strongly activate the phosphoinositide-3-kinase/Akt pathway independent of the generation of reactive oxygen species. Arch. Biochem. Biophys. 397: 232-9].

Based on these findings, and the teachings of WO99/40783 and WO 00/18885 which illustrate that copper chelators induce expansion of renewable stem cells from a variety of sources, the present inventors set out to test the suitability of cells expanded ex vivo in the presence of transition metal chelators, retinoic acid receptor antagonists and nicotinamide, and/or PI 3 kinase inhibitors in tissue regeneration therapy.

While reducing the present invention to practice and as is illustrated in the Examples section which follows, the present inventors uncovered that ex vivo expanded stem cells are highly suitable for tissue regeneration. In particular, the present inventors have shown that stem cells derived from hematopoietic or non-hematopoietic sources can be expanded in ex vivo long-term cultures supplemented with transition metal chelators, retinoic acid receptor antagonists, nicotinamide, and/or PI 3 kinase inhibitors and can further be transplanted into a recipient organ in which they ultimately trans-differentiate into other cell types characterizing the tissue of the recipient organ.

### SUMMARY OF THE INVENTION

According to one aspect of the present invention there is provided a method of in vivo differentiating stem cells into a target tissue, the method comprising: (a) obtaining a population of ex vivo expanded stem cells, the stem cells having been derived from the donor tissue; and (b) administering the stem cells to the target tissue, so as to induce differentiation of the stem cells into at least one cell type characterizing the target tissue.

According to another aspect of the present invention there is provided a method of treating an individual suffering from a disorder requiring cell or tissue replacement comprising: (a) subjecting isolated stem cells to culturing conditions selected suitable for inducing cell proliferation and suppressing cell differentiation, thereby obtaining an expanded stem cell population; and (b) introducing the expanded stem cell population into a tissue of the individual associated with the disorder thereby inducing differentiation of cells of the expanded stem cell population into cells characterizing the tissue, thereby treating the individual suffering from the disorder requiring cell or tissue replacement.

According to yet another aspect of the present invention there is provided a method of in-tissue differentiating adult stem cells into cells of a predetermined type comprising: (a) culturing the adult stem cells obtained from a donor tissue under conditions selected suitable for inducing cell proliferation and suppressing cell differentiation, thereby obtaining an expanded stem cell population; and (b) introducing the expanded adult stem cell population into a target tissue of a predetermined type to thereby differentiate the expanded stem cell population into cells characterizing the target tissue.

According to further features in preferred embodiments of the invention described below, the donor tissue has phenotypic and functional characteristics which are identical to those of the target tissue.

According to still further features in the described preferred embodiments the donor tissue has phenotypic and functional characteristics which are different from those of the target tissue.

According to still further features in the described preferred embodiments the stem cells derived from the donor tissue are selected from the group consisting of embryonic stem cells and neonatal and/or adult stem cells.

According to still further features in the described preferred embodiments the embryonic stem cells are selected from the group consisting of embryonic stem cells and embryonic germ cells.

According to still further features in the described preferred embodiments the neonatal and/or adult stem cells are selected from the group consisting of hematopoietic stem cells and non-hematopoietic stem cells.

According to still further features in the described preferred embodiments the hematopoietic stem cells are selected from the group consisting of bone marrow cells, neonatal umbilical cord blood cells and peripheral blood cells.

According to still further features in the described preferred embodiments the hematopoietic stem cells derived from the donor tissue are CD34+ enriched cells.

According to still further features in the described preferred embodiments the hematopoietic stem cells derived from the donor tissue are AC133+ enriched cells.

According to still further features in the described preferred embodiments the ex vivo expanded stem cells are characterized by down-regulated expression of cell surface antigens CD38, CD3, CD61, CD19, CD33, CD14, CD15 and/or CD4.

According to still further features in the described preferred embodiments the non-hematopoietic stem cells are selected from the group consisting of neuronal stem cell, neuronal progenitor cells, oligodendrocyte progenitors, mesenchymal stem cells, hepatocyte stem cells, liver stem cells, epidermal stem cells, cardiac stem cells.

According to still further features in the described preferred embodiments the stem cells derived from the donor tissue are mixed with committed cells.

According to still further features in the described preferred embodiments the stem cells derived from the donor tissue are obtained from a donor which is syngeneic, allogeneic and/or xenogeneic with respect to a subject having the target tissue.

According to still further features in the described preferred embodiments the target tissue comprises endodermal cells, ectodermal cells and/or mesodermal cells.

According to still further features in the described preferred embodiments said target tissue which comprises said endodermal cells is selected from the group consisting of pharynx, esophagus, stomach, intestines, liver, pancreas, trachea and lungs.

According to still further features in the described preferred embodiments said target tissue which comprises said ectodermal cells is selected from the group consisting of brain, adrenal gland, retina and epidermal skin.

According to still further features in the described preferred embodiments the target tissue which comprises mesodermal cells is selected from the group consisting of connective tissue, mesenchyme, bone, cartilage, muscle, fibrous tissue, dermal skin, heart, bone marrow and tubules of the urogenital system.

According to still further features in the described preferred embodiments the stem cells derived from the donor tissue are of an endodermal origin, an ectodermal origin and/or a mesodermal origin.

According to still further features in the described preferred embodiments the disorder is selected from the group consisting of a neurological disorder, a muscular disorder, a cardiovascular disorder, an hematological disorder, a skin disorder, a liver disorder and a pancreatic disorder.

According to still further features in the described preferred embodiments the obtaining the population of ex vivo expanded stem cells is effected by culturing stem cells under conditions suitable for inducing cell proliferation and suppressing cell differentiation.

According to still further features in the described preferred embodiments the conditions are selected capable of reducing an expression and/or activity of CD38 in the stem cells.

According to still further features in the described preferred embodiments the conditions further comprise providing the cells with nutrients and cytokines.

According to still further features in the described preferred embodiments the cytokines are early acting cytokines.

According to still further features in the described preferred embodiments the early acting cytokines are selected from the group comprising stem cell factor, FLT3 ligand, interleukin-1, interleukin-2, interleukin-3, interleukin-6, interleukin-10, interleukin-12, tumor necrosis factor-α and thrombopoietin.

According to still further features in the described preferred embodiments the cytokines are late acting cytokines.

According to still further features in the described preferred embodiments the late acting cytokines are selected from the group comprising granulocyte colony stimulating factor, granulocyte/macrophage colony stimulating factor, erythropoietin, FGF, EGF, NGF, VEGF, LIF, Hepatocyte growth factor and macrophage colony stimulating factor.

According to still further features in the described preferred embodiments the conditions comprise providing the cells with a transition metal chelator or chelate.

According to still further features in the described preferred embodiments the transition metal chelator or chelate is selected from the group consisting of polyamine chelating agents, ethylendiamine, diethylenetriamine, triethylenetetramine, triethylenediamine, tetraethylenepentamine, aminoethylethanolamine, aminoethylpiperazine, pentaethylenehexamine, triethylenetetramine-hydrochloride, tetraethylenepentamine-hydrochloride, pentaethylenehexamine-hydrochloride, tetraethylpentamine, captopril, penicilamine, N,N′-bis(3-aminopropyl)-1,3-propanediamine, N,N,Bis(2 animoethyl) 1,3 propane diamine, 1,7-dioxa-4,10-diazacyclododecane, 1,4,8,11-tetraaza cyclotetradecane-5,7-dione, 1,4,7-triazacyclononane trihydrochloride, 1-oxa-4,7,10-triazacyclododecane, 1,4,8,12-tetraaza cyclopentadecane, 1,4,7,10-tetraaza cyclododecane.

According to still further features in the described preferred embodiments the conditions selected capable of reducing the expression and/or activity of CD38 in the stem cells comprise an agent that downregulates CD38 expression.

According to still further features in the described preferred embodiments the agent that downregulates CD38 expression is selected from the group consisting of a retinoic acid receptor antagonist, a retinoid X receptor antagonist and a Vitamin D receptor antagonist.

According to still further features in the described preferred embodiments the retinoic acid receptor antagonist is selected from the group consisting of: AGN 194310; AGN 193109; 3-(4-Methoxy-phenylsulfanyl)-3-methyl-butyric acid; 6-Methoxy-2,2-dimethyl-thiochroman-4-one,2,2-Dimethyl-4-oxo-thiochroman-6-yltrifluoromethane-sulfonate; Ethyl 4-((2,2 dimethyl-4-oxo-thiochroman-6-yl)ethynyl)-benzoate; Ethyl 4-((2,2-dimethyl-4-triflouromethanensulfonyloxy-(2H)-thiochromen-6-yl)ethynyl)-benzoate(41); Thiochromen-6-yl]-ethynyl]-benzoate(yl); (p-[(E)-2-[3′4′-Dihydro-4,4′ dimethyl-7′-(heptyloxy)-2′H-1-benzothiopyran-6′yl] propenyl]benzoic acid 1′1′-dioxide; 2E,4E,6E-[7-(3,5-Di-t-butyl-4-n-butoxyphenyl)-3-methyl]-octa-2,4,6-trienoic acid; 2E,4E,6E-[7-(3,5-Di-t-butyl-4-n-propoxyphenyl)-3-methyl]-octa-2,4,6-trienoic acid; 2E,4E,6E-[7-(3,5-Di-t-butyl-4n-pentoxyphenyl)-3-methyl]-octa-2,4,6-trienoic acid; 2E,4E,6E-[7-(3,5-Di-t-butyl-4-n-hexoxyphenyl)-3-methyl]-octa-2,4,6-trienoic acid; 2E,4E,6E-[7-(3,5-Di-t-butyl-4-n-heptoxyphenyl)-3-methyl]-octa-2,4,6-trienoic acid; 2E,4E,6E-[7-(3,5-Di-t-butyl-4-n-octoxyphenyl)-3-methyl]-octa-2,4,6-trienoic acid; (2E,4E,6E)-7-[3-t-butyl-5-(1-phenyl-vinyl)-phenyl]-3-methyl-octa-2,4,6-trienoic acid; 2E,4E,6E-[7-(3,5-Di-t-butyl-4-{[4,5-³H₂]-n-pentoxy}phenyl)-3-methyl]-octa-2,4,6-trienoic acid; (2E,4E)-(1RS,2RS)-5-[2-(3,5-di-tert.butyl-2-ethoxy-phenyl)-cyclopropyl]-3-methyl-penta-2,4-dienoic acid ethyl ester; (2E,4E)-(1RS,2RS)-5-[2-(3,5-di-tert.butyl-2-ethoxy-phenyl)-cyclopropyl]-3-methyl-penta-2,4-dienoic acid; (2E,4E)-(1RS,2RS)-5-[2-(3,5-di-tert.butyl-2-butoxy-phenyl)-cyclopropyl)-3-methyl-penta-2,4-dienoic acid; (2E,4E,6Z)-7-[3,5-di-tert.butyl-2-ethoxyphenyl]3-methyl-2,4,6-octatrienoic acid; (2E,4E,6Z)-7-[3,5-di-tert.butyl-2-butyloxyphenyl]-3-methyl-2,4,6-octatrienoic acid; 4-(5,6,7,8-tetrahydro-5,5,8,8-tetramethyl-2-naphthalene-carboxamido)benzoic acid; (2E,4E)-3-methyl-5-[(1S,2S)-2-(5,5,8,8-tetramethyl-5,6,7,8-tetrahydro-naphthalen-2-yl)-cyclopropyl]-penta-2,4-dienoic acid; p-[(E)-2-[3′,4′-Dihydro-4′,4′-dimethyl-7′-(heptyloxy)-2′H-1-benzothiopyran-6′-yl)-propenyl]benzoic acid; 1′,1′-dioxide, 4-(7,7,10,10-Tetramethyl-1-pyridin-3-ylmethyl-4,5,7,8,9,10-hexahydro-1H-naphto[2,3-g]indol-3-yl)-benzoic acid; (2E,4E,6Z)-7-[3,5-di-tert.butyl-2-methoxyphenyl]-3-methyl-2,4,6-octatrienoic acid; (2E,4E,6Z)-7-[3,5-di-tert.butyl-2-ethoxyphenyl]-3-methyl-2,4,6-octatrienoic acid; (2E,4E,6Z)-7-[3,5-di-tert.butyl-2-hexyloxyphenyl]-3-methyl-2,4,6-octatrienoic acid; (2E,4E,6Z)-7-[3,5-di-tert.butyl-2-octyloxyphenyl]-3-methyl-2,4,6-octatrienoic acid; and (2E,4E)-(1RS,2RS)-5-[2-(3,5-di-tert-butyl-2-butoxy-phenyl)-cyclopropyl]-3-methyl-penta-2,4-dienoic acid, (2E,4E,6Z)-7-(3-n-propoxy-5,6,7,8-tetrahydro-5,5,8,8-tetramethylnaphthalene-2-yl)-3-methylocta-2,4,6-trienoic acid, and 4-(5H-2,3(2,5 dimethyl-2,5-hexano)-5-n-propyldibenzo[b,e][1,4]diazepin-11-yl)benzoic acid, 4-(5H-2,3-(2,5-dimethyl-2,5-hexano)-5methyl-8-nitrodibenzo[b,e][1,4]diazepin-11-yl)benzoic acid, 4-{[4-(4-Ethylphenyl)2,2-dimethyl-(2H)-thiochromen-6-yl]ethynyl}benzoic acid, 4-[4-2methyl-1,2-dicarba-closo-dodecaboran-1-yl-phenylcarbamoyl]benzoic acid, 4-[4,5,7,8,9,10-hexahydro-7,7,10,10-tetramethyl-1-(3-pyridylmethyl)-anthra[1,2-b]pyrrol-3-yl]benzoic acid, (3-pyridylmethyl)]5-thiaanthra[2,1-b]pyrrol-3-yl)benzoic acid, and (3-pyridylmethyl)-anthra[2 ml-d]pyrazol-3-yl]benzoic acid.

According to still further features in the described preferred embodiments the retinoid X receptor antagonist is selected from the group consisting of: LGN100572, LGN100574, 1-(3-hydroxy-5,6,7,8-tetrahydro-5,5,8,8-tetramethylnaphthalene-2-yl)ethanone, 1-(3-propoxy-5,6,7,8-tetrahydro-5,5,8,8-tetramethylnaphthalene-2-yl)ethanone, 3-(3-propoxy-5,6,7,8-tetrahydro-5,5,8,8-tetramethylnaphthalene-2-yl)but-2-enenitrile, 3-(3-propoxy-5,6,7,8-tetrahydro-5,5,8,8-tetramethylnaphthalene-2-yl)but-2-enal, (2E,4E,6E)-7-3[-propoxy-5,6,7,8-tetrahydro 5,5,8,8-tetramethyl-2-naphthalene-2-yl]-3-methylocta-2,4,6-trienoic acid, 4-[3,5,5,8,8-pentamethyl-5,6,7,8-tetrahydro-2-naphthyl)carbonyl]benzoic acid, 4-[1-(3,5,5,8,8-pentamethyl-5,6,7,8-tetrahydro-2-naphthyl)ethenyl]benzoic acid, 4-[1-(3,5,5,8,8-pentamethyl-5,6,7,8-tetrahydro-2-naphthyl)cyclopropyl]benzoic acid, 4-[1-(3,5,5,8,8-pentamethyl-5,6,7,8-tetrahydro-2-naphthyl)ethenyl]benzenete trazole, 2-[1-(5,5,8,8-tetramethyl-5,6,7,8-tetrahydro-2-naphthyl)ethenyl)pyridine-5-carboxylic acid, 2-[1-(3,5,5,8,8-pentamethyl-5,6,7,8-tetrahydro-2-naphthyl)ethyl]pyridine-5-carboxylic acid, ethyl-2-[1-(3,5,5,8,8-pentamethyl-5,6,7,8-tetrahydro-2-naphthyl)ethenyl]pyridine-5-carboxylate, 5-[1-3,5,5,8,8-pentamethyl-5,6,7,8-tetrahydro-2-naphthyl)ethenyl]pyridine-2-carboxylic acid, 2-[1-(3,5,5,8,8-pentamethyl-5,6,7,8-tetrahydro-2-naphthyl)cyclopropyl]pyridine-5-carboxylic acid, methyl 2-[1-(3,5,5,8,8-pentamethyl-5,6,7,8-tetrahydro-2-naphthyl)cyclopropyl]pyridine-5-carboxylate, 4-[1-(3,5,5,9,8-pentamethyl-5,6,7,8-tetrahydro-2-naphthyl)ethenyl]-N-(4-hydroxyphenyl)benzamide, 2-[1-(3,5,5,8,8-Pentamethyl-5,6,7,8-tetrahydro-2-naphthyl)ethenyl]pyridine-5-carboxylic acid, 2-[1-(3,5,5,8,8-Pentamethyl-5,6,7,8-tetrahydro-2-naphthyl)cyclopropyl]pyridine-5-carboxylic acid, 4-[(3,5,5,8,8-pentamethyl-5,6,7,8-tetrahydro-2-naphthyl)carbonyl]benzoic acid butyloxime, 4-[(3,5,5,8,8-pentamethyl-5,6,7,8-tetrahydro-2-naphthyl)carbonyl]benzoic acid propyloxime, 4-[(3,5,5,8,8-pentamethyl-5,6,7,8-terrahydro-2-naphthyl)carbonyl]benzoic acid cyanoimine, 4-[(3,5,5,8,8-pentamethyl-5,6,7,8-tetrahydro-2-naphthyl)carbonyl]benzoic acid allyloxime, 4-[(3,5,5,8,8-pentamethyl-5,6,7,8-tetrahydro-2-naphthyl)carbonyl]benzoic acid 4-(3-methylbut-2-enoic acid)oxime, and 4-[(3,5,5,8,8-pentamethyl-5,6,7,8-tetrahydro-2-naphthyl)carbonyl]benzoic acid 1-aminoethyloxime, (2E,4E,6Z)-7-(3-n-propoxy-5,6,7,8-tetrahydro-5,5,8,8-tetramethylnaphthalene-2-yl)-3-methylocta-2,4,6-trienoic acid, 4-(5H-2,3(2,5 dimethyl-2,5-hexano)-5-n-propyldibenzo[b,e][1,4]diazepin-11-yl)benzoic acid, and 4-(5H-2,3-(2,5-dimethyl-2,5-hexano)-5methyl-8-nitrodibenzo[b,e][1,4]diazepin-11-yl)benzoic acid.

According to still further features in the described preferred embodiments the Vitamin D receptor antagonist is selected from the group consisting of: 1 alpha, 25-(OH)-D3-26,23 lactone; 1alpha, 25-dihydroxyvitamin D (3); the 25-carboxylic ester ZK159222; (23S)-25-dehydro-1 alpha-OH-D (3); (23R)-25-dehydro-1 alpha-OH-D (3); 1 beta, 25 (OH)₂ D₃; 1 beta, 25(OH)₂-3-epi-D₃; (23S) 25-dehydro-1 alpha(OH) D3-26,23-lactone; (23R) 25-dehydro-1 alpha(OH)D3-26,23-lactone and Butyl-(5Z,7E,22E-(1S,7E,22E-(1S,3R,24R)-1,3,24-trihydroxy-26,27-cyclo-9,10-secocholesta-5,7,10(19),22-tetraene-25-carboxylate).

According to still further features in the described preferred embodiments the agent that downregulates CD38 expression is an antagonist for reducing a capacity of the stem cells in responding to retinoic acid, retinoid and/or Vitamin D.

According to still further features in the described preferred embodiments the agent that downregulates CD38 expression is a polynucleotide.

According to still further features in the described preferred embodiments the polynucleotide encodes an anti CD38, an anti retinoic acid receptor, an anti retinoid X receptor or an anti Vitamin D receptor intracellular antibody.

According to still further features in the described preferred embodiments the polynucleotide encodes an anti CD38, an anti retinoic acid receptor, an anti retinoid X receptor or an anti Vitamin D receptor antibody.

According to still further features in the described preferred embodiments the polynucleotide is a small interfering polynucleotide molecule directed to cause intracellular CD38, retinoic acid receptor, retinoid X receptor or Vitamin D receptor mRNA or gene degradation.

According to still further features in the described preferred embodiments the small interfering polynucleotide molecule is selected from the group consisting of an RNAi molecule, an anti-sense molecule, a ribozyme molecule and a DNAzyme molecule.

According to still further features in the described preferred embodiments the agent that downregulates CD38 expression is a PI 3-kinase activity or expression inhibitor.

According to still further features in the described preferred embodiments the PI 3-kinase expression inhibitor is a polynucleotide.

According to still further features in the described preferred embodiments the polynucleotide encodes a PI 3-kinase intracellular antibody.

According to still further features in the described preferred embodiments the polynucleotide encodes a PI 3-kinase antibody.

According to still further features in the described preferred embodiments the polynucleotide is a small interfering polynucleotide molecule directed to cause intracellular PI 3-kinase mRNA or gene degradation.

According to still further features in the described preferred embodiments the small interfering polynucleotide molecule is selected from the group consisting of an RNAi molecule, an anti-sense molecule, a ribozyme molecule and a DNAzyme molecule.

According to still further features in the described preferred embodiments the polynucleotide is a DNA vector containing a dominant negative PI 3-kinase construct.

According to still further features in the described preferred embodiments the PI 3-kinase activity inhibitor is selected from the group consisting of wortmannin and LY294002.

According to still further features in the described preferred embodiments the conditions selected capable of reducing the expression and/or activity of CD38 in the stem cells comprise an agent that inhibits CD38 activity.

According to still further features in the described preferred embodiments the agent that inhibits CD38 activity is nicotinamide, a nicotinamide analog, a nicotinamide or a nicotinamide analog derivative or a nicotinamide or a nicotinamide analog metabolite.

According to still further features in the described preferred embodiments the nicotinamide analog is selected from the group consisting of benzamide, nicotinethioamide, nicotinic acid and α-amino-3-indolepropionic acid.

According to still further features in the described preferred embodiments the conditions are selected capable of reducing a capacity of the stem cells to respond to retinoic acid.

According to still further features in the described preferred embodiments the conditions comprise nicotinamide, a nicotinamide analog, a nicotinamide or a nicotinamide analog derivative or a nicotinamide or a nicotinamide analog metabolite.

According to still further features in the described preferred embodiments the conditions comprise a PI 3-kinase activity or expression inhibitor.

According to still further features in the described preferred embodiments the conditions are selected capable of reducing a capacity of the stem cells to respond to retinoids.

According to still further features in the described preferred embodiments the conditions are selected capable of reducing a capacity of the stem cells to respond to Vitamin D.

According to still further features in the described preferred embodiments the conditions are selected capable of reducing a capacity of the stem cells to respond to signaling pathways involving retinoic acid receptor.

According to still further features in the described preferred embodiments the conditions are selected capable of reducing a capacity of the stem cells to respond to signaling pathways involving retinoid X receptor.

According to still further features in the described preferred embodiments the conditions are selected capable of reducing a capacity of the stem cells to respond to signaling pathways involving Vitamin D receptor.

The present invention successfully addresses the shortcomings of the presently known configurations by providing methods of differentiating ex vivo expanded stem cells in-tissue and in vivo, and methods of treating disorders using ex vivo expanded stem cells.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are photomicrographs of Hematoxylin-Eosin stained frozen rat heart sections of rats transplanted with CD34+ cells via a left ventricular (LV) cavity infusion. Note the area with atypical cells originating from transplanted CD34+ cells situated near the myocardial infarction (MI) scar loci (marked with arrows). Magnifications are X200 (FIG. 1a) and X400 (FIG. 1b).

FIGS. 2a-c are photomicrographs of FISH analyses using probes specific to the human X and Y chromosomes. Shown are FISH analyses of cytospin samples of ex vivo expanded CD34+ cells derived from a pool of male and female newborn cord blood samples (FIG. 2a, magnification is X1000), and of rat heart sections derived from rats transplanted with human CD34+ cells (FIGS. 2b and c, magnifications are X1000). The green and orange hybridization signals correspond to the X and Y human chromosomes, respectively. Nuclei are stained with DAPI. Note that while rat heart cells derived from transplanted human stem cells are labeled with green and orange hybridization signals (FIG. 2b, cells marked with red arrows), endogenous rat heart cells are unlabeled (FIG. 2b, cells marked with yellow arrows).

FIGS. 3a-b illustrate human derived rat heart cells in AC133+ transplanted rats. Three weeks following transplantation, frozen rat heart sections were subjected to FISH analysis using fluorescent probes specific to the human X and Y chromosomes. Note the green and orange hybridization signals corresponding to the human X and Y chromosomes, respectively, in rat heart cells (FIG. 3a) and in cells localized around a blood vessel (FIG. 3b). Nuclei are stained with DAPI. Magnification is X1000.

FIG. 4 illustrates an immuno-fluorescence analysis using anti von-Willebrand factor antibody followed by a FISH analysis using probes specific for human X and Y chromosomes. Shown is a frozen rat heart section with CD34+ engrafted cord blood cells expressing the endothelial von-Willebrand factor marker in the cytoplasm (green labeling, marked with arrows) and the X and Y chromosomes hybridization signals in the nuclei. Magnification is X1000.

FIG. 5 is a photomicrograph of an immunohistochemistry analysis of cytospin samples of CD34+ ex vivo expanded cord blood cells using an anti HLA-DR antibody. HLA-DR positive cells are labeled with a dark brown staining. Magnification is X400.

FIG. 6 is a photomicrograph of an immunohistochemistry staining of rat bone marrow cells using an anti HLA-DR antibody. Rat bone marrow cells derived from human AC133+ transplanted cells (IV infused) are labeled with a brown staining corresponding to HLA-DR expression. Magnification is X400.

FIGS. 7a-b are photomicrographs of an immunohistochemistry staining of frozen rat heart sections using an anti HLA-DR antibody. Note the brown labeling corresponding to HLA-DR expression in cells derived from human AC133+ transplanted cells. Magnifications are X200 (FIG. 7a) and X400 (FIG. 7b).

FIGS. 8a-b are photomicrographs of an immunohistochemistry staining of frozen lung parenchyma sections. Note the brown labeling corresponding to HLA-DR expression in cells derived from human AC133+ transplanted cells.

### DESCRIPTION OF THE PREFERRED EMBODIMENTS

The present invention is of (i) methods of differentiating ex vivo expanded stem cells in-tissue and in vivo; and (ii) methods of treating individuals suffering from a disorder by cell replacement or tissue replacement therapy using ex vivo expanded stem cells.

The principles and operation of the methods according to the present invention may be better understood with reference to the accompanying descriptions and examples.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Pluripotent human stem cells provide biomedical research with new approaches for drug development and testing, and for organ repair and replacement since it has been shown that such cells can be used for the replacement of dysfunctional or degenerative tissue.

Tissue replacement can use stem cells derived from embryonic, fetal or adult tissues.

The recent discoveries that hematopoietic stem cells can give rise to non-hematopoietic tissues such as neuronal and cardiac tissues suggest that these cells may have greater differentiation potential than was previously assumed and thus opens new frontiers for therapeutic applications using such cells [Lu D et al. (2002) Intravenous administration of human umbilical cord blood reduces neurological deficit in the rat after traumatic brain injury. Cell Transplant. 11:275-81].

However, expansion of hematopoietic stem cells presents numerous challenges and thus limits the use of such cells in applications where relatively large numbers of such cells are necessary, such as tissue repair and cell replacement. Most of hematopoietic stem cell culturing protocols practiced to date use various combinations of early and late cytokines which enable stem cell proliferation for a limited time period, following which, the stem cells go through differentiation steps and commit to distinct hematopoietic cell lineage [Koller M R, et al. Large-scale expansion of human stem and progenitor cells from bone marrow mononuclear cells in continuous perfusion cultures. (1993). Blood, 82: 378; Haylock D N, et al. Ex vivo expansion and maturation of peripheral blood CD34+ cells into the myeloid lineage. (1992). Blood, 80: 1405; Brugger W, et al. Ex vivo expansion of enriched peripheral blood CD34+progenitor cells by stem cell factor, interleukin-1 beta (IL-1 beta), IL-6, IL-3, interferon-gamma, and erythropoietin. (1993). Blood, 81: 2579; Sato N, et al. In-vitro expansion of human peripheral blood CD34+ cells. (1993). Blood, 82:3600].

Presently practiced stem cell culturing methods are ill suited for generating large numbers of pluripotent stem cells since such methods cannot retain harvested stem cells in a proliferative undifferentiated state for prolonged periods of time. In order to do so, stem cell differentiation must be reversibly inhibited or delayed and stem cells self-renewal prolonged.

This is particularly important in the case of adult stem cells which are typically recovered in small quantities and are difficult to maintain in a non-differentiated yet proliferative state.

Such cells are of importance both in commercial and research applications due to the restrictions now placed on use and generation of embryonic stem cells.

In addition, prior art methodology has failed to adequately teach methods of inducing stem cell differentiation and in particular adult stem cell differentiation into predetermined cell types.

While reducing the present invention to practice, the present inventors uncovered that ex vivo expanded stem cells, particularly, ex vivo expanded adult stem cells, can differentiate in vivo following administration. The present inventors have also uncovered that such cells target into a large variety of tissues in response to injury.

Therefore, according to one aspect of the present invention there is provided a method of inducing in vivo differentiation of ex vivo expanded stem cells. The method is effected by first obtaining a population of ex vivo expanded stem cells (described in detail hereinbelow) and then administering the expanded stem cell population in a tissue, so as to induce differentiation of the stem cells into at least one cell type characterizing the tissue.

As is further described hereinabove, stem cells of the present invention can be of any type including embryonic stem cells and neonatal (fetal) and/or adult stem cells. The latter include stem cells from both hematopoietic origin (e.g., bone marrow cells, neonatal umbilical cord blood cells and peripheral blood cells) and non-hematopoietic origin (e.g., neuronal stem cell, neuronal progenitor cells, oligodendrocyte progenitors, mesenchymal stem cells, hepatocyte stem cells, liver stem cells, epidermal stem cells, cardiac stem cells).

Preferably, the stem cells are derived from a first tissue (also referred to herein as “donor tissue”) and administered into a second tissue (also referred to herein as “target tissue”). The first and second tissues are preferably of a different type, in which case differentiation is termed herein as “trans-differentiation”. However, it should be noted that the first and second tissues can be of the same type, in which case differentiation is termed herein as “cis-differentiation”.

As is described hereinbelow and in the examples section which follows, the expanded stem cell population administered into a tissue is preferably characterized by downregulated expression of cell surface antigens such as CD38, CD3, CD61, CD19, CD33, CD14, CD15 and CD4.

Various approaches can be utilized to generate an expanded stem cell population characterized by downregulated expression of some or preferably all of the cell surface antigens described above. The following section provides examples of suitable methodology.

Ex Vivo Expansion of Stem Cells in the Presence of Transitional Metal Chelators

As described in the Background section hereinabove, transition metal chelators, and especially, copper chelators, can inhibit differentiation of stem and progenitor cells, thereby prolonging cell proliferation and expansion ex vivo. As is futher described in U.S. patent application Ser. No. 09/986,897, ex vivo expansion of CD34+ cells in the presence of the copper chelator, tetraethylenepentamine, TEPA, and high or low doses of early-acting cytokines or a combination of early and late acting cytokines resulted in significant increases of in cell cloning efficiency and percentage of CD34+ cells.

Thus, according to one preferred embodiment of the present invention, ex vivo expansion of stem cells is effected in the presence of nutrients, cytokines and transition metal chelators.

Cytokines of the present invention can be early or late acting cytokines and can be purchased from any cytokines vendor such as Pepro Tech, Inc., Rocky Hill, N.J., and Cytokines from R&D, Minneapolis, Minn. Examples for early acting cytokines include, but are not limited to, stem cell factor, FLT3 ligand, interleukin-1, interleukin-2, interleukin-3, interleukin-6, interleukin-10, interleukin-12, tumor necrosis factor-α and thrombopoietin.

Examples of late acting cytokines include, but are not limited to, granulocyte colony stimulating factor, granulocyte/macrophage colony stimulating factor, erythropoietin, FGF, EGF, NGF, VEGF, LIF, Hepatocyte growth factor and macrophage colony stimulating factor.

As used herein, the phrase “transition metal chelator” refers to a transition metal ligand that has at least two atoms capable of coordinating with an indicated metal, so as to form a ring. A transition metal chelator of an indicated transition metal, is free of, i.e., not complexed with, an ion of the indicated transition metal and hence, the phrase “copper chelator”, for example, refers to a chelator of copper, which is free of, i.e., not complexed with, a copper ion.

Such chelators, can be for example, polyamine chelating agents, ethylendiamine, diethylenetriamine, triethylenetetramine, triethylenediamine, tetraethylenepentamine, aminoethylethanolamine, aminoethylpiperazine, pentaethylenehexamine, triethylenetetramine-hydrochloride, tetraethylenepentamine-hydrochloride, pentaethylenehexamine-hydrochloride, tetraethylpentamine, captopril, penicilamine, N,N′-bis(3-aminopropyl)-1,3-propanediamine, N,N,Bis (2 animoethyl) 1,3 propane diamine, 1,7-dioxa-4,10-diazacyclododecane, 1,4,8,11-tetraaza cyclotetradecane-5,7-dione, 1,4,7-triazacyclononane trihydrochloride, 1-oxa-4,7,10-triazacyclododecane, 1,4,8,12-tetraaza cyclopentadecane, 1,4,7,10-tetraaza cyclododecane. These transition metal chelators are commercially available from Novasep (France).

As described in PCT/IL/03/00062 ex vivo expansion of stem cell was also achieved in the presence of transition metal chelates.

As used herein the phrase “transition metal chelate” refers to a chelator of an indicated transition metal which is complexed with an ion of the indicated transition metal and hence, the phrase “copper chelate”, for example, refers to a chelator of copper complexed with a copper ion.

Ex Vivo Expansion of Stem Cells While Down-Regulating CD38 Activity or Expression

Regulation of CD38 is considered a critical event in stem cell development and controls the step leading to self-renewal or differentiation. Experiments combining different reagents, active at different cellular targets, demonstrated neither additive nor synergistic effect. These results supported the role of the CD38 protein as a causative factor in regulation of stem cells self-renewal.

As is described in the Background section hereinabove, most of the hematopoietic pluripotent stem cells which are capable of self-renewal and multi-lineage differentiation are CD34+/CD38−.

As is further described in the Background section hereinabove, retinoic acid receptor (RAR)-mediated signaling results in induction of expression of the differentiation marker CD38 cell surface antigen whereas antagonists to RAR downregulate CD38 expression.

Moreover, as disclosed in PCT/IL03/00064, nicotinamide, the CD38 inhibitor, represses the process of differentiation of stem cells and stimulates and prolongs the phase of active cell proliferation and expansion ex vivo. In addition, chemical agents such as antagonists of the RAR, RXR and VDR also repress the process of differentiation of stem cells and stimulates and prolongs, for up to 16-18 weeks, the phase of active cell proliferation and expansion ex vivo.

PCT/IL03/00064 also discloses that primary hepatocyte cultures incubated with agents such as retinoic acid receptor antagonists of the RAR and RXR super families, are characterized by an increased proportion of cells producing α-fetoprotein.

Altogether, these data suggest that ex vivo expansion of stem cells, of hematopoietic and other origins, can be achieved using various agents which interfere with CD38 expression and/or activity and thereby induce ex vivo and/or in vivo expansion of stem cell populations. Thus application of such agents to, for example, hematopoietic stem cells, would result in large numbers of undifferentiated CD34+/Lin− (CD33, CD14, CD15, CD4, etc.), as well as CD34+/CD38− cells, especially CD34+_{dim}/Lin− cells.

As is further described in the Background section hereinabove, inhibitors of PI 3-kinase are also capable of preventing or downregulating CD38 mRNA expression.

Moreover, downstream signal transduction imposed by nuclear receptors such as the RARs, RXRs, VDRs and THRs may also be abrogated by inhibition of PI 3-kinase, which is an obligatory factor for proper receptor signaling.

In addition, as is further described in the Background section hereinabove, cellular PI 3-kinase activity is strongly enhanced after exposure to Cu⁺⁺ [Ostrakhovitch E A et al. (2002). Copper ions strongly activate the phosphoinositide-3-kinase/Akt pathway independent of the generation of reactive oxygen species. Arch. Biochem. Biophys. 397: 232-9].

Considering the above data, it may be postulated that RA and Vitamin D enhance cell differentiation via induction of dimerization of the nuclear receptors, RAR and RXR and RXR and VDR, respectively, which, following activation, recruit PI 3-kinase since downstream signal transduction by the nuclear heterodimers appears to be PI 3-kinase depended. Only in the presence of the active form of PI 3-kinase, these receptors will further control gene expression and as a result, will induce and accelerate cell differentiation. Thus, inhibition of PI 3-kinase enzymatic activity by, for example, site specific PI 3-kinase inhibitors, will downregulate CD38 expression, as is demonstrated by abrogation of leukemic cell differentiation induced by either RA or Vitamin D.

Compounds that specifically inhibit RA and Vitamin D induction in late stages of differentiation of leukemic cells, as well as down-regulate CD38 expression, i.e., RAR, RXR and VDR antagonists, will also inhibit cytokine induction in stern/early progenitor cell differentiation. Therefore, inhibition of PI 3-kinase activity and/or expression by site specific inhibitors will result in inhibition of CD34+ cell differentiation, similar or better than that exhibited by RAR antagonists and/or nicotinamide.

The proposed activity of PI 3-kinase also correlates with the effect of copper ion concentration on proliferation or differentiation of stem cells, suggesting that copper modulates cell proliferation and differentiation via activation (at high intracellular copper content) or deactivation (at low intracellular copper content) of PI 3-kinase which is an obligatory factor in up regulation of CD38 gene expression and cell differentiation.

Under low copper content (imposed by supplementing the culture media with a copper chelator such as tetraethylenepentamine (TEPA) PI 3-kinase is less active, resulting in a delay in cell differentiation. On the other hand, at high cell copper content, PI 3-kinase is strongly activated, resulting in acceleration of cell differentiation.

Thus, as is clearly illustrated in the above cited references, site-specific reagents, such as the RAR antagonists (which downregulate CD38 gene expression), nicotinamides (which abrogates CD38 enzymatic activity), as well as agents capable of reducing the enzymatic activity of PI 3-kinase (directly or by reduction of cell copper content which results in reduced or abolished signal transduction via retinoid receptors), are capable of inhibiting CD34+ cell differentiation and thus can be utilized in expansion of stem cell cultures.

It should be noted that reducing the capacity of stem cells to respond to the above described signaling pathways is reversible. It has been previously shown that following a period of 16-18 weeks in culture cells exposed to pathway inhibitors ceased to expand and started to differentiate thus indicating that cells expanded using the methods described hereinabove do not transform into other cell lines but rather retain their ability to differentiate into one of several predetermined cell lines.

Examples of signaling pathway inhibitors which can be utilized with the present invention include, but are not limited to, retinoic acid receptor antagonists such as, AGN 194310; AGN 193109; 3-(4-Methoxy-phenylsulfanyl)-3-methyl-butyric acid; 6-Methoxy-2,2-dimethyl-thiochroman-4-one,2,2-Dimethyl-4-oxo-thiochroman-6-yltrifluoromethane-sulfonate; Ethyl 4-((2,2 dimethyl-4-oxo-thiochroman-6-yl)ethynyl)-benzoate; Ethyl 4-((2,2-dimethyl-4-triflouromethanensulfonyloxy-(2H)-thiochromen-6-yl)ethynyl)-benzoate(41); Thiochromen-6-yl]-ethynyl]-benzoate(yl); ([(E)-2-[3′4′-Dihydro-4,4′-dimethyl-7′-(heptyloxy)-2′H-1-benzothiopyran-6′yl]propenyl]benzoic acid 1′1′-dioxide; 2E,4E,6E-[7-(3,5-Di-t-butyl-4-n-butoxyphenyl)-3-methyl]-octa-2,4,6-trienoic acid; 2E,4E,6E-[7-(3,5-Di-t-butyl-4-n-propoxyphenyl)-3-methyl]-octa-2,4,6-trienoic acid; 2E,4E,6E-[7-(3,5-Di-t-butyl-4-n-pentoxyphenyl)-3-methyl)-octa-2,4,6-trienoic acid; 2E,4E,6E-[7-(3,5-Di-t-butyl-4-n-hexoxyphenyl)₃-methyl]-octa-2,4,6-trienoic acid; 2E,4E,6E-[7-(3,5-Di-t-butyl-4-n-heptoxyphenyl)-3-methyl]-octa-2,4,6-trienoic acid; 2E,4E,6E-[7-(3,5-Di-t-butyl-4-n-octoxyphenyl)-3-methyl]-octa-2,4,6-trienoic acid; (2E,4E,6E)-7-(3-t-butyl-5-(1-phenyl-vinyl)-phenyl]-3-methyl-octa-2,4,6-trienoic acid; 2E,4E,6E-[7-(3,5-Di-t-butyl-4-{[4,5-³H₂]-n-pentoxy}phenyl)-3-methyl]-octa-2,4,6-trienoic acid; (2E,4E)-(1RS,2RS)-5-[2-(3,5-di-tert.butyl-2-ethoxy-phenyl)-cyclopropyl]-3-methyl-penta-2,4-dienoic acid ethyl ester; (2E,4E)-(1RS,2RS)-5-[2-(3,5-di-tert.butyl-2-ethoxy-phenyl)-cyclopropyl]-3-methyl-penta-2,4-dienoic acid; (2E,4E)-(1RS,2RS)-5-[2-(3,5-di-tert.butyl-2-butoxy-phenyl)-cyclopropyl]-3-methyl-penta-2,4-dienoic acid; (2E,4E,6Z)-7-[3,5-di-tert.butyl-2-ethoxyphenyl]3-methyl-2,4,6-octatrienoic acid; (2E,4E,6Z)-7-[3,5-di-tert.butyl-2-butyloxyphenyl]-3-methyl-2,4,6-octatrienoic acid; 4-(5,6,7,8-tetrahydro-5,5,8,8-tetramethyl-2-naphthalene-carboxamido) benzoic acid; (2E,4E)-3-methyl-5-[(1S,2S)-2-(5,5,8,8-tetramethyl-5,6,7,8-tetrahydro-naphthalen-2-yl)-cyclopropyl]-penta-2,4-dienoic acid; p-[(E)-2-[3′,4′-Dihydro-4′,4′-dimethyl-7′-(heptyloxy)-2′-1-benzothiopyran-6′-yl]propenyl]benzoic acid; 1′,1′-dioxide, 4-(7,7,10,10-Tetramethyl-1-pyridin-3-ylmethyl-4,5,7,8,9,10-hexahydro-1H-naphto[2,3-g]indol-3-yl)-benzoic acid; (2E,4E,6Z)-7-[,5-di-tert.butyl-2-methoxyphenyl]-3-methyl-2,4,6-octatrienoic acid; (2E,4E,6Z)-7-[3,5-di-tert.butyl-2-ethoxyphenyl]-3-methyl-2,4,6-octatrienoic acid; (2E,4E,6Z)-7-[3,5-di-tert.butyl-2-hexyloxyphenyl]-3-methyl-2,4,6-octatrienoic acid; (2E,4E,6Z)-7-[3,5-di-tert.butyl-2-octyloxyphenyl]-3-methyl-2,4,6-octatrienoic acid; and (2E,4E)-(1RS,2RS)-5-[2-(3,5-di-tert-butyl-2-butoxy-phenyl)-cyclopropyl]-3-methyl-penta-2,4-dienoic acid, (2E,4E,6Z)-7-(3-n-propoxy-5,6,7,8-tetrahydro-5,5,8,8-tetramethylnaphthalene-2-yl)-3-methylocta-2,4,6-trienoic acid, and 4-(5H-2,3(2,5 dimethyl-2,5-hexano)-5-n-propyldibenzo[b,e][1,4]diazepin-11-yl)benzoic acid, 4-(5H-2,3-(2,5-dimethyl-2,5-hexano)-5methyl-8-nitrodibenzo[b,e][,4]diazepin-11-yl)benzoic acid, 4-{[4-(4-Ethylphenyl)2,2-dimethyl-(2H)-thiochromen-6-yl]ethynyl}benzoic acid, 4-[4-2methyl-1,2-dicarba-closo-dodecaboran-1-yl-phenylcarbamoyl]benzoic acid, 4-[4,5,7,8,9,10-hexahydro-7,7,10,10-tetramethyl-1-(3-pyridylmethyl)-anthra[1,2-b]pyrrol-3-yl]benzoic acid, (3-pyridylmethyl)-]5-thiaanthra[2,1-b]pyrrol-3-yl)benzoic acid, and (3-pyridylmethyl)-anthra[2 ml-d]pyrazol-3-yl]benzoic acid.

Retinoid X receptor antagonists of the present invention include, but are not limited to, LGN100572, LGN100574, 1-(3-hydroxy-5,6,7,8-tetrahydro-5,5,8,8-tetramethylnaphthalene-2-yl)ethanone, 1-(3-propoxy-5,6,7,8-tetrahydro-5,5,8,8-tetramethylnaphthalene-2-yl)ethanone, 3-(3-propoxy-5,6,7,8-tetrahydro-5,5,8,8-tetramethylnaphthalene-2-yl)but-2-enenitrile, 3-(3-propoxy-5,6,7,8-tetrahydro-5,5,8,8-tetramethylnaphthalene-2-yl)but-2-enal, (2E,4E,6E)-7-3 [-propoxy-5,6,7,8-tetrahydro 5,5,8,8-tetramethyl-2-naphthalene-2-yl]-3-methylocta-2,4,6-trienoic acid, 4-[3,5,5,8,8-pentamethyl-5,6,7,8-tetrahydro-2-naphthyl)carbonyl]benzoic acid, 4-[1-(3,5,5,8,8-pentamethyl-5,6,7,8-tetrahydro-2-naphthyl)ethenyl]benzoic acid, 4-[1(3,5,5,8,8-pentamethyl-5,6,7,8-tetrahydro-2-naphthyl)cyclopropyl]benzoic acid, 4-[1-(3,5,5,8,8-pentamethyl-5,6,7,8-tetrahydro-2-naphthyl)ethenyl]benzenete trazole, 2-[1-(5,5,8,8-tetramethyl-5,6,7,8-tetrahydro-2-naphthyl)ethenyl]pyridine-5carboxylic acid, 2-[1-(3,5,5,8,8-pentamethyl-5,6,7,8-tetrahydro-2-naphthyl)ethyl]pyridine-5-carboxylic acid, ethyl-2-[1-(3,5,5,8,8-pentamethyl-5,6,7,8-tetrahydro-2-naphthyl)ethenyl]pyridine-5-carboxylate, 5-[1-3,5,5,8,8-pentamethyl-5,6,7,8-tetrahydro-2-naphthyl)ethenyl]pyridine-2-carboxylic acid, 2-[1-(3,5,5,8,8-pentamethyl-5,6,7,8-tetrahydro-2-naphthyl)cyclopropyl]pyridine-5-carboxylic acid, methyl 2-[1-(3,5,5,8,8-pentamethyl-5,6,7,8-tetrahydro-2-naphthyl)cyclopropyl]pyridine-5-carboxylate, 4-[1-(3,5,5,8,8-pentamethyl-5,6,7,8-tetrahydro-2-naphthyl)ethenyl]-N-(4-hydroxyphenyl)benzamide, 2-[1-(3,5,5,8,8-Pentamethyl-5,6,7,8-tetrahydro-2-naphthyl)ethenyl]pyridine-5-carboxylic acid, 2-[1-(3,5,5,8,8-Pentamethyl-5,6,7,8-tetrahydro-2-naphthyl)cyclopropyl]pyridine-5-carboxylic acid, 4-[(3,5,5,8,8-pentamethyl-5,6,7,8-tetrahydro-2-naphthyl)carbonyl]benzoic acid butyloxime, 4-[(3,5,5,8,8-pentamethyl-5,6,7,8-tetrahydro-2-naphthyl)carbonyl]benzoic acid propyloxime, 4-[(3,5,5,8,8-pentamethyl-5,6,7,8-terrahydro-2-naphthyl)carbonyl]benzoic acid cyanoimine, 4-[(3,5,5,8,8-pentamethyl-5,6,7,8-tetrahydro-2-naphthyl)carbonyl]benzoic acid allyloxime, 4-[(3,5,5,8,8-pentamethyl-5,6,7,8-tetrahydro-2-naphthyl)carbonyl]benzoic acid 4-(3-methylbut-2-enoic acid)oxime, and 4-[(3,5,5,8,8-pentamethyl-5,6,7,8-tetrahydro-2-naphthyl)carbonyl]benzoic acid 1-aminoethyloxime, (2E,4E,6Z)-7-(3-n-propoxy-5,6,7,8-tetrahydro-5,5,8,8-tetramethylnaphthalene-2-yl)-3-methylocta-2,4,6-trienoic acid, 4-(5H-2,3(2,5 dimethyl-2,5-hexano)-5-n-propyldibenzo[b,e][1,4]diazepin-11-yl)benzoic acid, and 4-(5H-2,3-(2,5-dimethyl-2,5-hexano)-5methyl-8-nitrodibenzo[b,e][1,4]diazepin-11-yl)benzoic acid.

Vitamin D receptor antagonists of the resent invention include, but are not limited to, 1 alpha, 25-(OH)-D3-26,23 lactone; 1alpha, 25-dihydroxyvitamin D (3); the 25-carboxylic ester ZK159222; (23S)-25-dehydro-1 alpha-OH-D (3); (23R)-25-dehydro-1 alpha-OH-D (3); 1 beta, 25 (OH)₂ D₃; 1 beta, 25(OH)₂-3-epi-D₃; (23S) 25-dehydro-1 alpha(OH) D3-26,23-lactone; (23R) 25-dehydro-1 alpha(OH)D3-26,23-lactone and Butyl-(5Z,7E,22E-(1S,7E,22E-(1S,3R,24R)-1,3,24-trihydroxy-26,27-cyclo-9,10-secocholesta-5,7,10(19),22-tetraene-25-carboxylate).

The above listed antagonists are known for their high affinity towards their respective cognate receptors and as such can be utilized in low concentrations. In addition to the above, it should be noted that anti PI 3-kinase, anti retinoic acid receptor, anti retinoid X receptor and/or anti Vitamin D receptor antibodies or antibody fragments can also be used to specifically inhibit activity of these receptors while antisense or siRNA molecules directed at sequences encoding such receptors can be utilized to inhibit expression thereof. These approaches are further described hereinbelow.

It will be appreciated that direct inhibitors of CD38 activity or expression, can also be utilized as potent inhibitors of stem cell differentiation. Inhibition of CD38 enzymatic activity can be effected using antibodies or specific blockers (e.g., substrate analogs), while CD38 transcription or translation can be downregulated using antisense molecules, siRNA. These approaches are further described hereinbelow. Any of these approaches will all result in inhibition of CD34+ cell differentiation and increase in ex vivo expansion of early progenitor cells.

Antibodies

The term “antibody” as used in this invention includes intact molecules as well as functional fragments thereof, such as Fab, F(ab′)₂, and Fv that are capable of binding to macrophages.

These functional antibody fragments are defined as follows:

Fab, the fragment which contains a monovalent antigen-binding fragment of an antibody molecule, can be produced by digestion of whole antibody with the enzyme papain to yield an intact light chain and a portion of one heavy chain; Fab′, the fragment of an antibody molecule that can be obtained by treating whole antibody with pepsin, followed by reduction, to yield an intact light chain and a portion of the heavy chain; two Fab′ fragments are obtained per antibody molecule; (Fab′)₂, the fragment of the antibody that can be obtained by treating whole antibody with the enzyme pepsin without subsequent reduction; F(ab′)₂ is a dimer of two Fab′ fragments held together by two disulfide bonds;

Fv, defined as a genetically engineered fragment containing the variable region of the light chain and the variable region of the heavy chain expressed as two chains; and Single chain antibody (“SCA”), a genetically engineered molecule containing the variable region of the light chain and the variable region of the heavy chain, linked by a suitable polypeptide linker as a genetically fused single chain molecule.

Methods of producing such antibody fragments are known in the art. (See for example, Harlow and Lane, Antibodies: A Laboratory Manual, Cold Spring Harbor Laboratory, New York, 1988, incorporated herein by reference).

Antibody fragments according to the present invention can be prepared by expression in E. Coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. Coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins recipient antibody in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies [Cole et al., Monoclonal Antibodies and Cancer Therapy, Alan R Liss, p. 77 (1985) and Boerner et al., J. Immunol., 147(1):86-95 (1991)]. Similarly, human can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

Recombinant Antibodies

Polynucleotides encoding anti CD38, RAR, RXR, VDR or PI 3-kinase antibodies can be expressed using prokaryotic or eukaryotic expression systems.

To generate such an expression vector, polynucleotide segments encoding anti CD38, RAR, RXR, VDR or PI 3-kinase antibodies, devoid of extracellular secretion signal peptide sequence, are ligated into, for example, a commercially available expression vector system suitable for transforming mammalian cells and for directing the expression of the antibodies within the transformed cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides, etc.

Suitable mammalian expression vectors for use with the present invention include, but are not limited to, pcDNA3, pcDNA3.1(+/−), pZeoSV2(+/−), pSecTag2, pDisplay, pEF/myc/cyto, pCMV/myc/cyto, pCR3.1, which are available from Invitrogen, pCI which is available from Promega, pBK-RSV and pBK-CMV which are available from Strategene, pTRES which is available from Clontech, and their derivatives.

Viral expression vectors can be particularly useful for introducing an anti CD38, RAR, RXR, VDR or PI 3-kinase antibody polynucleotide into a cell (see, for example U.S. Pat. No. 5,399,346). Viral vectors provide the advantage that they can infect host cells with relatively high efficiency and can infect specific cell types.

The expression vector described above can be delivered into host cells using a variety of delivery approaches, including, but not limited to, microinjection, electroporation, liposomes, epidermal patches, iontophoresis or receptor-mediated endocytosis. The selection of a particular method will depend, for example, on the cell into which the polynucleotide is to be introduced, as well as whether the cell is isolated in culture, or is in a tissue or organ in culture or in situ.

Antisense Polynucleotides

Antisense molecules for inhibiting expression of the receptors described above can be expressed in target stem cells or preferably directly provided thereto (e.g., as an additive to a culture medium). In any case, design of antisense molecules which can be used to efficiently inhibit receptor expression must take into account sequence specificity and in the case of oligonucleotides which are provided directly to the cells, various delivery strategies.

The prior art teaches of a number of delivery strategies which can be used to efficiently deliver oligonucleotides into a wide variety of cell types (see, for example, Luft (1998) J Mol Med 76(2): 75-6; Kronenwett et al. (1998) Blood 91(3): 852-62; Rajur et al. (1997) Bioconjug Chem 8(6): 935-40; Lavigne et al. (1997) Biochem Biophys Res Commun 237(3): 566-71 and Aoki et al. (1997) Biochem Biophys Res Commun 231(3): 540-5).

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. (1999) Biotechnol Bioeng 65(1): 1-9].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al. (1998) Nature Biotechnology 16, 1374-1375).

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

In order to ensure efficient inhibition oligonucleotide analogs need to be devised in a suitable manner.

For example, problems arising in connection with double-stranded DNA (dsDNA) recognition through triple helix formation have been diminished by a clever “switch back” chemical linking, whereby a sequence of polypurine on one strand is recognized, and by “switching back”, a homopurine sequence on the other strand can be recognized. Also, good helix formation has been obtained by using artificial bases, thereby improving binding conditions with regard to ionic strength and pH.

In addition, in order to improve half-life as well as membrane penetration, a large number of variations in polynucleotide backbones have been done.

Oligonucleotides can be modified either in the base, the sugar or the phosphate moiety. These modifications include, for example, the use of methylphosphonates, monothiophosphates, dithiophosphates, phosphoramidates, phosphate esters, bridged phosphorothioates, bridged phosphoramidates, bridged methylenephosphonates, dephospho internucleotide analogs with siloxane bridges, carbonate bridges, carboxymethyl ester bridges, carbonate bridges, carboxymethyl ester bridges, acetamide bridges, carbamate bridges, thioether bridges, sulfoxy bridges, sulfono bridges, various “plastic” DNAs, anomeric bridges and borane derivatives (Cook, 1991, Anti-Cancer Drug Design 6: 585).

International patent application WO 89/12060 discloses various building blocks for synthesizing oligonucleotide analogs, as well as oligonucleotide analogs formed by joining such building blocks in a defined sequence. The building blocks may be either “rigid” (i.e., containing a ring structure) or “flexible” (i.e., lacking a ring structure). In both cases, the building blocks contain a hydroxy group and a mercapto group, through which the building blocks are said to join to form oligonucleotide analogs. The linking moiety in the oligonucleotide analogs is selected from the group consisting of sulfide (—S—), sulfoxide (—SO—), and sulfone (—SO2—).

International patent application WO 92/20702 describes an acyclic oligonucleotide which includes a peptide backbone on which any selected chemical nucleobases or analogs are stringed and serve as coding characters as they do in natural DNA or RNA. These new compounds, known as peptide nucleic acids (PNAs), are not only more stable in cells than their natural counterparts, but also bind natural DNA and RNA 50 to 100 times more tightly than the natural nucleic acids cling to each other. PNA oligomers can be synthesized from the four protected monomers containing thymine, cytosine, adenine and guanine by Merrifield solid-phase peptide synthesis. In order to increase solubility in water and to prevent aggregation, a lysine amide group is placed at the C-terminal region.

Typically, antisense mediated inhibition targets mRNA, although a second option for disrupting gene expression at the level of transcription uses synthetic oligonucleotides capable of hybridizing with double stranded DNA. A triple helix is formed. Such oligonucleotides may prevent binding of transcription factors to the gene's promoter and therefore inhibit transcription. Alternatively, they may prevent duplex unwinding and, therefore, transcription of genes within the triple helical structure.

siRNAs

Another mechanism of down regulating enzymes at the transcript level is RNA interference (RNAi), an approach which utilizes small interfering dsRNA (siRNA) molecules that are homologous to the target mRNA and lead to its degradation [Carthew, 2001, Curr Opin Cell Biol 13(2):244-8]. RNAi is an evolutionarily conserved surveillance mechanism that responds to double-stranded RNA by sequence-specific silencing of homologous genes (Fire et al., 1998, Nature 391, 806-811; Zamore et al., 2000, Cell 101, 25-33). RNAi is initiated by the dsRNA-specific endonuclease dicer, which promotes cleavage of long dsRNA into double-stranded fragments between 21 and 25 nucleotides long, termed small interfering RNA (siRNAs) (Zamore et al., 2000, Cell 101, 25-33; Elbashir et al., 2001, Genes Dev. 15, 188-200; Hammond et al., 2000, Nature 404, 293-296; Bernstein et al., 2001, Nature 409, 363-366). siRNA are incorporated into a protein complex that recognizes and cleaves target mRNAs (Nykanen et al., 2001, Cell 107, 309-321).

RNAi has been increasingly used for the sequence-specific inhibition of gene expression. The possibility of interfering with any specific target RNA has rendered RNAi a valuable tool in both basic research and therapeutic applications. RNAi was first used for gene silencing in nematodes (Fire et al., 1998, Nature 391, 806-811).

Recent scientific publications have validated the efficacy of such short double stranded RNA molecules in inhibiting target mRNA expression and thus have clearly demonstrated the inhibitory potential of such molecules. For example, RNAi has been utilized to inhibit expression of hepatitis C (McCaffrey et al., 2002, Nature 418, 38-39), HIV-1 (Jacque et al., 2002, Nature 418, 435-438), cervical cancer cells (Jiang and Milner 2002, Oncogene 21, 6041-8) and leukemic cells (Wilda et al., 2002, Oncogene 21, 5716-24).

Several parameters must be taken into account when designing RNAi for inhibiting expression in mammalian cells. Since the introduction of dsRNA into mammalian cells does not result in efficient Dicer-mediated generation of siRNA (Caplen et al., 2000, Gene 252, 95-105; Ui-Tei et al., 2000, FEBS Lett. 479, 79-82) short siRNA duplexes of typically 21 to 25-base pairs are utilized to initiate target cleavage.

Such siRNA molecules can be chemically synthesized as 21 to 25-nucleotide siRNA duplexes (Elbashir et al., 2001, Genes Dev. 15, 188-200; McCaffrey et al., 2002, Nature 418, 38-39). Synthetic siRNA oligonucleotide duplexes can be prepared with either ribonucleotide 3′ overhangs or with deoxyribonucleotide 3′ overhangs (Hohjoh 2002, FEBS Lett. 521, 195-9). They can also be prepared as a sense-stranded DNA/antisense-stranded RNA hybrids or vise versa.

The siRNA used by the present invention can also be transcribed in vitro from plasmids and administered into the stem cells. Transcripts that include two self-complementary siRNAs annealed to form a loop region can be further processed by single-stranded ribonucleases and/or other proteins into a functional duplex siRNA molecule (Leirdal and Sioud, 2002, Biochem Biophys Res Commun 295, 744-8). siRNA can also be prepared from dsRNA by Escherichia Coli RNase III cleavage into endoribonuclease-prepared siRNA (esiRNA).

Ribozymes

Ribozymes are being increasingly used for the sequence-specific inhibition of gene expression by the cleavage of mRNAs encoding proteins of interest. The possibility of designing ribozymes to cleave any specific target RNA has rendered them valuable tools in both basic research and therapeutic applications. Novel ribozymes can be designed to cleave known substrate, using either random variants of a known ribozyme or random-sequence RNA as a starting point (Pan, T. and Uhlenbeck, O. C. Biochemistry 1996; 31: 3887; Tsang, J. and Joyce, G. F. Biochemistry 1994; 33: 5966; Breaker, R. R. and Joyce, G. Chemistry and Biology 1994; 1: 223). However, ribozymes may be susceptible to hydrolysis within the cells, sometimes limiting their inhibitory application.

DNAzymes

Recently, a new class of catalytic molecules called “DNAzymes” was created (Breaker, R. R. and Joyce, G. Chemistry and Biology 1995, 2: 655; Santoro, S. W. & Joyce, G. F. Proc. Natl, Acad. Sci. USA 1997, 943: 4262). DNAzymes are single-stranded molecules which specifically cleave target mRNA molecules. A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for review of DNAzymes see Khachigian, L M Curr Opin Mol Ther 2002,4: 119-21).

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann. Meeting Am. Soc. Gen. Ther. www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

Although the above described approaches are capable of maintaining stem cells in a proliferative, non-differentiated state for extended periods of time, thus enabling accumulation of large amounts of pluripotent cells, the effect of such stem cell expansion approaches is reversible, thus allowing in-tissue differentiation of expanded stem cells populations following administration thereof.

Thus, following ex vivo expansion, the stem cells of the present invention are administered into a tissue. Such administration is preferably effected in vivo, although ex vivo administration of the cells into an explant is also contemplated herein, especially in cases where development of the stem cells is to be monitored for research purposes. In the latter case, the tissue explant utilized is preferably cultured under conditions suitable for retaining explant morphology and physiology.

As used herein the term “tissue” refers to a cellular mass composed of one or more specific cell types organized into a specific architecture and optionally function. As mentioned hereinabove, the tissue of the present invention can be isolated (explant) or it can form a part of a subject (a mammal).

As is further described in the Examples section which follows, ex vivo expanded neonatal umbilical cord blood stem cells were administered in vivo into myocardial infarctions (MI) of SCID nude rats. Three weeks following stem cells transplantation, administered cells differentiated in vivo into bone marrow cells, myocardial cells, vascular cells and lung parenchyma cells.

The expanded stem cells can be derived from a donor which is syngeneic, allogeneic and/or xenogeneic with respect to the source of the target tissue.

As is further demonstrated in the Examples section which follows, the present inventor unexpectedly discovered that ex vivo expanded stem cells differentiate into various cell type, including heart, lung, bone marrow and vascular cells following in vivo administration.

Depending on the source stem cells and target organ, differentiation can be either cis-differentiation or trans-differentiation or a combination of both.

As is mentioned hereinabove “cis-differentiation” refers to differentiation of stem cells into a tissue identical to the tissue from which they were derived. For example, the differentiation of CD34+ hematopoietic cells to different committed/mature blood cells constitutes cis-differentiation.

As is mentioned hereinabove “trans-differentiation” refers to differentiation of stem cells into a tissue distinct from which they were derived. For example, the differentiation of CD34+hematopoietic cells to cells of different tissue origin, e.g., cardiac cells, constitutes trans-differentiation.

As is mentioned hereinabove, administration of the expanded stem cells is either effected in vitro, or in vivo. In vivo administration of stem cells is effected by a direct administration of the cells into the tissue or by an indirect administration of the cell into a blood vessel feeding the target tissue (preferably carrying oxygenated blood), using any suitable route. Preferred administration routes include, but are not limited to, in-tissue injection, infusion, transfusion, perfusion and the like. Please see the Examples section for further description of one preferred administration method.

Since the expanded stem cells of the present invention are capable of differentiating in vivo into a variety of specific cell types, and since differentiation can be predetermined according to source and target tissue combinations, the method of the present invention can be utilized in cell replacement therapy.

Results provided herein demonstrate the potential of stem cells, expanded and administered using the methods described hereinabove, to regenerate damaged tissue and to be used in cell replacement therapy. As is further demonstrated in the Examples section which follows, transplantation of cord blood stem cells into MI rats resulted in cell differentiation and horning of differentiated cells to loci of an MI scar and injured lung parenchyma.

Thus, the present methodology can be used in treating disorders which require cell or tissue replacement.

The disorder can be a neurological disorder, a muscular disorder, a cardiovascular disorder, an hematological disorder, a skin disorder, a liver disorder, and the like.

Myeln disorders form an important group of human neurological diseases that are, as yet, incurable. Progress in animal models, particularly in transplanting cells of the oligodendrocyte lineage, has resulted in significant focal re-myelination and physiological evidence of restoration of function (Repair of myelin disease: Strategies and progress in animal models. Molecular Medicine Today. 1997. pp. 554-561). Future therapies could involve both transplantation and promotion of endogenous repair, and the two approaches could be combined with ex vivo manipulation of donor tissue.

Defects in cartilage and bones can also be treated using the teachings of the present invention. Methods of utilizing stem cells for treating such disorders are provided in U.S. Pat. No. 4,642,120.

Skin regeneration of a wound or burn in an animal or human can also be treated using the teachings of the present invention. Methods of utilizing stem cells for treating such disorders are provided in U.S. Pat. No. 5,654,186 and U.S. Pat. No. 5,716,411.

As used herein, the term “treating” refers to inhibiting or arresting the development of a disease, disorder or condition and/or causing the reduction, remission, or regression of a disease, disorder or condition in an individual suffering from, or diagnosed with, the disease, disorder or condition. Those of skill in the art will be aware of various methodologies and assays which can be used to assess the development of a disease, disorder or condition, and similarly, various methodologies and assays which can be used to assess the reduction, remission or regression of a disease, disorder or condition.

In addition to the above described application, the teachings of the present invention can also be utilized in several other therapeutic applications.

Transplantation of hematopoietic cells has become the treatment of choice for a variety of inherited or malignant diseases. While early transplantation procedures utilized the entire bone marrow (BM) population, recently, more defined populations, enriched for stem cells (CD34+ cells) have been used (Van Epps D E, et al. Harvesting, characterization, and culture of CD34+ cells from human bone marrow, peripheral blood, and cord blood. Blood Cells 20:411, 1994). In addition to bone marrow, such cells could also be derived from other sources such as peripheral blood (PB) and neonatal umbilical cord blood (CB) (Emerson S G. Ex-vivo expansion of hematopoietic precursors, progenitors, and stem cells: The next generation of cellular therapeutics. Blood 87:3082, 1996). Compared to BM, transplantation with PB cells shortens the period of pancytopenia and reduces the risks of infection and bleeding (Brugger W, et al. Reconstitution of hematopoiesis after high-dose chemotherapy by autologous progenitor cells generated in-vivo. N Engl J Med 333: 283, 1995; Williams S F, et al. Selection and expansion of peripheral blood CD34+ cells in autologous stem cell transplantation for breast cancer. Blood 87: 1687, 1996; Zimmerman R M, et al. Large-scale selection of CD34+peripheral blood progenitors and expansion of neutrophil precursors for clinical applications. J Hematotherapy, 5: 247, 1996).

An additional advantage of using PB for transplantation is its accessibility, although to date the limiting factor in PB transplantation stems from the low number of circulating pluripotent stem/progenitor cells available for harvesting.

To obtain enough PB-derived stem cells for transplantation, these cells are “harvested” by repeated leukophoresis following their mobilization from the marrow into the circulation by treatment with chemotherapy and cytokines. Such treatment is obviously not suitable for normal donors.

Thus, the use of ex vivo expanded stem cells for transplantation provides several advantages: (i) it reduces the volume of blood required for reconstitution of an adult hematopoietic system and may obviate the need for mobilization and leukophoresis; (ii) it enables storage of small number of PB or CB stem cells for potential future use; and (iii) it traverses contamination limitations often associated with autologous transplantation of recipients with malignancies. In such cases, contaminating tumor cells in autologous infusion often contribute to the recurrence of the disease, selecting and expanding CD34+ stem cells will reduce the load of tumor cells in the final transplant.

In addition, expanded stem cell cultures are depleted of T lymphocytes, and thus are advantageous in allogeneic transplants in which T-cells contribute to graft-versus-host disease (Koller M R, Emerson S G, Palsson B O. Large-scale expansion of human stem and progenitor cells from bone marrow mononuclear cells in continuous perfusion cultures. Blood 82:378, 1993; Lebkowski J S, et al. Rapid isolation and serum-free expansion of human CD34+ cells. Blood Cells 20: 404, 1994).

Clinical studies indicate that transplantation of ex vivo expanded cells derived from a small number of PB CD34+ cells can restore hematopoiesis in recipients treated with high doses of chemotherapy, although the results do not yet allow firm conclusions about long term in vivo hematopoietic capabilities of these cultured cells.

For successful transplantation, shortening the duration of the cytopenic phase, as well as long-term engraftment, is crucial. Inclusion of intermediate and late progenitor cells in the transplant could accelerate the production of donor-derived mature cells thereby shortening the cytopenic phase.

It is thus important, in such applications that ex-vivo expanded cells include, in addition to stem cells, more differentiated progenitor cells in order to optimize short-term recovery and long-term restoration of hematopoiesis. Expansion of intermediate and late progenitor cells, especially those committed to the neutrophilic and megakaryocytic lineages, concomitant with expansion of stem cells, should serve this purpose (Sandstrom C E, et al. Effects of CD34+ cell selection and perfusion on ex vivo expansion of peripheral blood mononuclear cells. Blood 86: 958, 1995).

Such cultures may be useful in restoring hematopoiesis in recipients with completely ablated bone marrow, as well as in providing a supportive measure for shortening recipient bone marrow recovery following conventional radio- or chemo-therapies.

In addition to the above, the teachings of the present invention can also be applied towards hepatic regeneration, muscle regeneration, and stimulation of bone growth for applications in osteoporosis.

The teachings of the present invention can also be applied to cases which require enhanced immune response or replacement of deficient functions, such as. for example, adoptive immunotherapy, including immunotherapy of various malignancies, immuno-deficiencies, viral and genetic diseases [Freedman A R, et al. Generation of T lymphocytes from bone marrow CD34+ cells in vitro. (1996). Nature Medicine. 2: 46; Heslop H E, et al. Long term restoration of immunity against Epstein-Barr virus infection by adoptive transfer of gene-modified virus-specific T lymphocytes. (1996) Nature Medicine, 2: 551; Protti M P, et al. Particulate naturally processed peptides prime a cytotoxic response against human melanoma in vitro. (1996). Cancer Res., 56: 1210].

The teachings of the present invention can also be used in gene therapy. Genetically modified and ex vivo expanded stem cells expressing stably integrated transgenes can be utilized in various gene therapy applications since such expanding stem cell populations are highly amenable to viral based transformations and thus can provide an excellent platform for any application which requires expression of therapeutic proteins.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### EXAMPLE 1

### Trans-Differentiation of CD34+ and AC133+ Cells into Rat Myocardium

To test the ability of ex vivo expanded CD34+ and/or AC133+ stem cells to trans-differentiate in recipient tissues, cord blood derived stem cells were expanded in the presence of copper chelators and cytokines, and transplanted into nude rats.

Materials and Experimental Methods

Sample collection and processing—Samples were obtained from umbilical human cord blood and were processed within 12 hours. Blood cells were mixed with 3% Gelatin (Sigma, St. Louis, Mo.) and allowed to sediment for 30 minutes to remove most red blood cells. The leukocyte-rich fraction was harvested, layered on a Ficoll-Hypaque column (density 1.077 gram/ml; Sigma) and centrifuged at 400×g for 30 minutes at room temperature. The mononuclear cells in the interface layer were then collected, washed three times in phosphate-buffered saline (PBS; Biological Industries, Beth Ha'Emek, Israel), and re-suspended in PBS solution containing 0.5% bovine serum albumin (BSA, Fraction V; Sigma).

Enrichment of CD34+ cells—CD34+ cells were purified from the mononuclear cell fraction using two cycles of immuno-magnetic separation using the miniMACS or Clinimax® CD34+ Progenitor Cell Isolation Kit (Miltenyi-Biotec, Auburn, Calif.) according to manufacturer's recommendations. The purity of the CD34+ cells obtained ranged between 95% and 98%, based on Flow Cytometry evaluation (FACStar^{plus} flow cytometer, Becton-Dickinson, Immunofluorometry systems, Mountain View, Calif.).

Enrichment of AC133+ cells—AC133+ cells were purified from the mononuclear cell fraction using two cycles of immuno-magnetic separation using the MiniMACS® direct CD133 cell isolation kit, human or Clinimax® 133 microbeads (Miltenyi-Biotec, Auburn, Calif.) according to manufacturer's recommendations. The purity of the AC133+ cells obtained ranged between 95% and 98%, based on Flow Cytometry evaluation (FACStar^{plus} flow cytometer).

Ex vivo expansion of CD34+ cells—Enriched CD34+ cell fractions were cultured in Teruflex T-150 transfer bags (Terumo Corp, Japan) with alpha minimal essential medium supplemented with 10% fetal bovine serum (FBS, Biological Industries), at about 10⁴ cells/ml medium. The media were further supplemented with the following human recombinant cytokines (all obtained from Pepro Tech, Inc., Rocky Hill, N J): Thrombopoietin (TPO), 50 ng/ml; interleukin-6 (IL-6), 50 ng/ml; FLT-3 ligand, 50 ng/ml; SCF, 50 ng/ml, in the presence or absence of a copper chelator (TEPA.5 HCl, Novasep, France) and replaced weekly. Cell cultures were incubated at 37° C. in an atmosphere of 5% CO₂ in air with extra humidity.

Ex vivo expansion of AC133+ cells—Enriched AC133+ cell fractions were cultured in Teruflex T-150 transfer bags (Terumo Corp., Japan) in alpha medium supplemented with 10% fetal bovine serum (Biological Industries) at about 10⁴ cells/ml medium. The media were further supplemented with the following human recombinant cytokines (all obtained from Cytokines from R&D, Minneapolis, Minn., or from Pepro Tech, Inc. Rocky Hill, N J): Thrombopoietin (TPO), 50 ng/ml; interleukin-6 (IL-6), 50 ng/ml; FLT-3 ligand, SCF, 50 ng/ml, in the presence or absence of a copper chelator (TEPA.5 HCl, Novasep, France) and was replaced weekly. Cell cultures were incubated at 37° C. in an atmosphere of 5% CO₂ in air with extra humidity.

Following three weeks in cultures, the CD34+ and/or AC133+ cells were re-selected using the respective magnetic beads and were further used for transplantation.

Myocardial Infarction (MI) rat model—The left anterior descending coronary artery of a Rowett (rnu/rnu) athymic nude rat (Harlan Laboratories, Ltd, Israel) was either ligated or was left intact (sham procedure).

Transfer of CD34+ cells into myocardial infarction rats—CD34+ cells were transferred to rat myocardium seven days following myocardial infarction using either one of the following two methods: (1) Rats were anesthetized and the chest was opened under sterile conditions. CD34+ cells (9×10⁶), or culture medium alone, were injected into the infarcted area, visualized by the surface scar and wall motion akinesis, using a 27-gauge needle. Following injections, the surgical incision was sutured closed. (2) Rats were anesthetized and placed in a supine position. CD34+ cells (9×10⁶) were aspirated into a scalp vein set (Vasuflo®) and a left ventricular (LV) cavity infusion was performed under the guidance of an echocardiography system (Sonos 5500, Hewlett Packard, USA) equipped with a 12.5-MHz phased-array transducer. The transducer was placed above the left side of the chest, and the cells were infused within 30 seconds using the 23-gauge needle of a scalp vein set which was gently introduced into the LV via the 4^{th} parastemal intercostal space.

Transfer of AC133+ cells into rat venous—AC133+ cells were transferred intra-venously (IV) according to Hayamizu K et al., 1998 (Monocyte-derived dendritic cell precursors facilitate tolerance to heart allografts after total lymphoid irradiation. Transplantation, 66: 1285-91) and Lu D et al., 2002 (Intravenous administration of human umbilical cord blood reduces neurological deficit in the rat after traumatic brain injury, Cell Transplant 11: 275-81).

Transthoracic echocardiography—was performed as a blind-experiment by an experienced technician using a commercially available echocardiography system equipped with 12.5-MHz phased-array transducer (Hewlett Packard, USA). Baseline echocardiogram was determined five days following MI and was compared to echocardiograms measured 1-3 weeks following implantation. All measurements were averaged for 3 consecutive cardiac cycles.

Fluorescent in situ hybridization (FISH)—Cord blood cytospin slides or frozen rat heart sections were subjected to two-color fluorescent in situ hybridization (FISH) using probes specific to the human X and Y chromosomes essentially as described elsewhere [Taneja K L et al., (2001). Multicolor fluorescence in situ hybridization with peptide nucleic acid probes for enumeration of specific chromosomes in human cells. Genes Chromosomes Cancer 30: 57-63; Heng H H, Tsui L C (1994). FISH detection on DAPI-banded chromosomes. Methods Mol Biol 33: 35-49; Banerjee S K et al., (1998). Quick-FISH: a rapid fluorescence in situ hybridization technique for molecular cytogenetic analysis. Biotechniques 24: 826-30; Fischer K et al., (1997). Fluorescence in-situ hybridization (FISH). Method and application. Med. Klin. 92: 279-83]. Slides were further subjected to nuclei staining using 4′, 6-diamino-2-phenylindole (DAPI, Sigma, USA) according to manufacturer's instructions.

Immunohistochemistry (IHC) and morphological analyses of trans-differentiated rat hearts—Rat hearts were harvested 1-3 weeks following transplantation and frozen sections were prepared for staining. For morphological analysis slides were stained with Hematoxylin-Eosin (Sigma, USA) according to manufacturer's instructions. IHC was performed using anti von-Willebrand factor antibody (Serotec, UK) or anti HLA-DR antibody (Serotec, UK) following manufacturer's instructions.

Experimental Results

Implantation of ex vivo generated stem cells into damaged rat hearts—CD34+ or AC133+ were re-selected from ex vivo expanded stem cells cultures and were transplanted into nude rats. One group of rats was transplanted with CD34+ cells by a direct injection into the myocardium infarcted scar (n=2) or into the normal myocardium tissue (n=2). Another group of rats was transplanted with CD34+ cells by an LV cavity transfusion into an MI-treated rat (n=1) or into a normal sham-treated rat (n=1). AC133+ cells were transplanted into rats by an intravenous (IV) infusion (n=2). One MI-treated rat served as a control and was injected with saline.

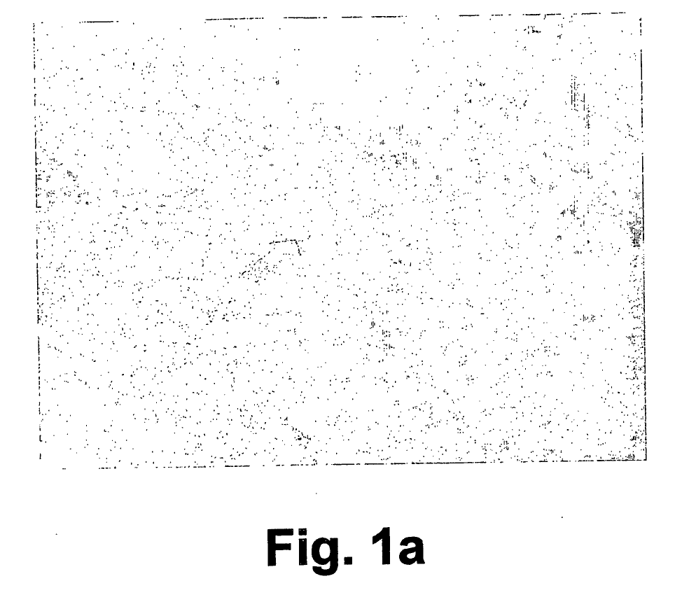

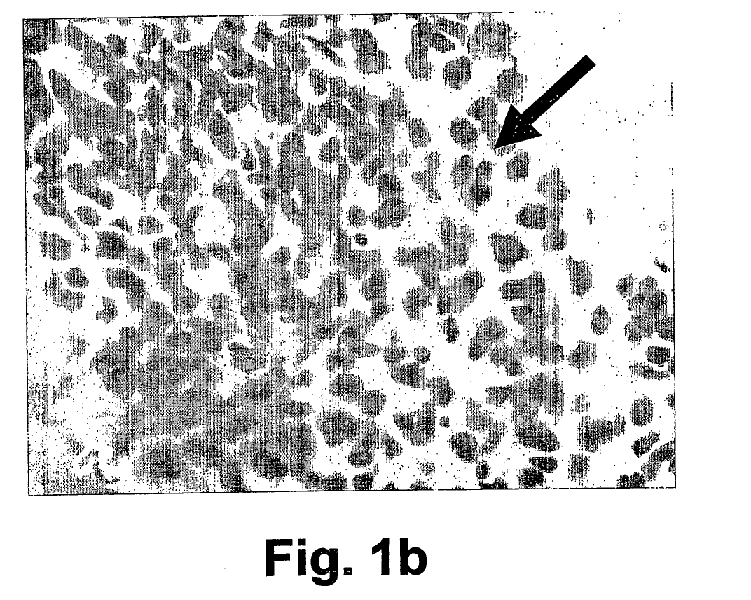

Donor stem cells colonized in hearts of treated rats—Three weeks following stem cells transplantation rat heart sections were prepared. In 5 out of 6 CD34+ transplanted rats the hearts were colonized by the donor stem cells. In the LV-treated rats, the donor cells homed to site of the scar and gave rise to clusters of cells that occupied loci situated near the scar (FIGS. 1a, b, arrow).

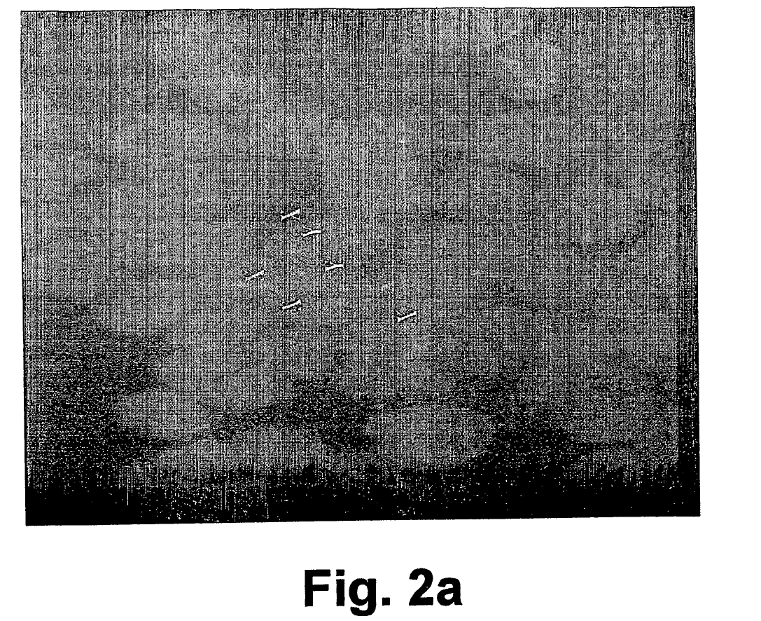

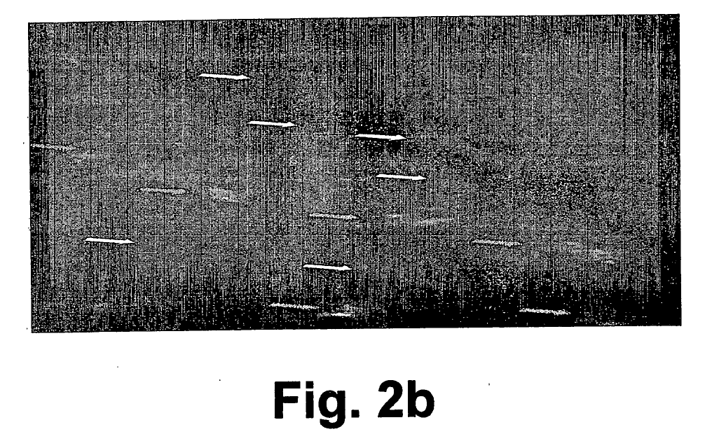

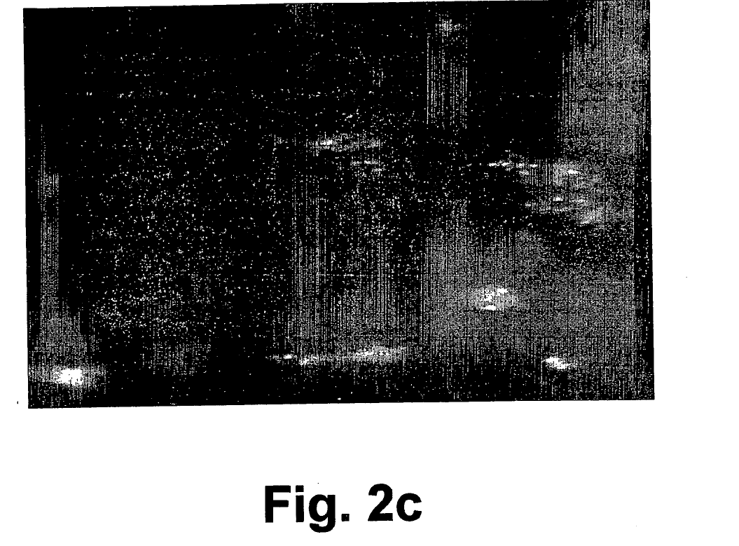

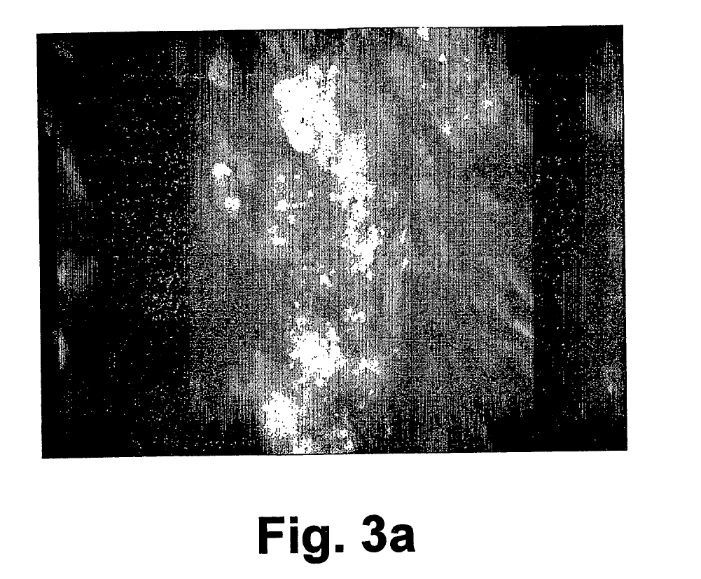

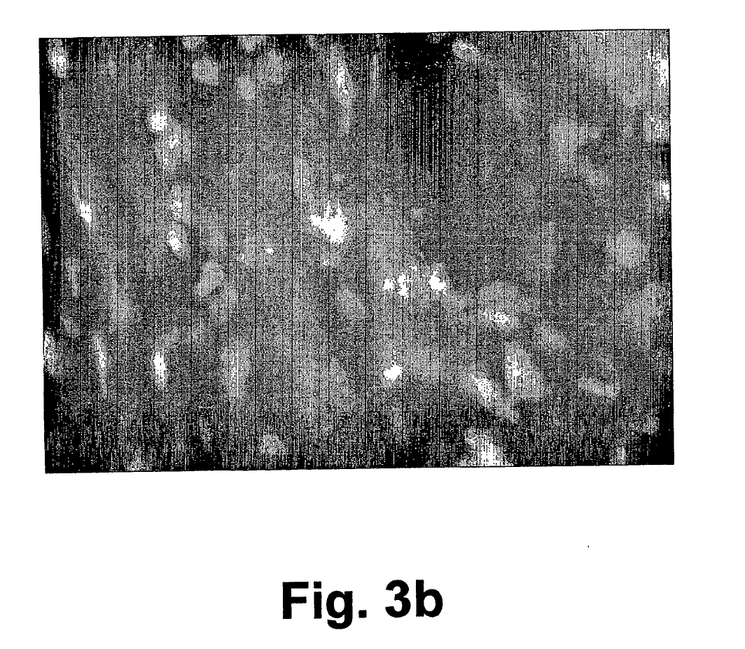

FISH analysis using probes specific to the human X and Y chromosomes demonstrated the male and female origin of CD34+cord blood cells expanded ex vivo (FIG. 2a). Three weeks following stem cells transplantation, FISH analyses of rat heart sections further confirmed the presence of human-originated cells in CD34+ (FIGS. 2b, c, arrows) or AC133+ (FIGS. 3a, b, fluorescent cells) transplanted rat hearts.

Trans-differentiation of stem cells into vascular cells—To evaluate the ability of transplanted stem cells to trans-differentiate in the recipient rats, rat heart sections were subjected to a fluorescent immunohistochemistry analysis using the endothelial cell marker, von-Willebrand factor, followed by a FISH analysis using human probes specific to the X and Y chromosomes. As is shown in FIG. 4 (arrows), the von-Willebrand factor was expressed in the cytoplasm of human-derived transplanted rat heart cells, demonstrating trans-differentiation of engraved stem cells into vascular cells.

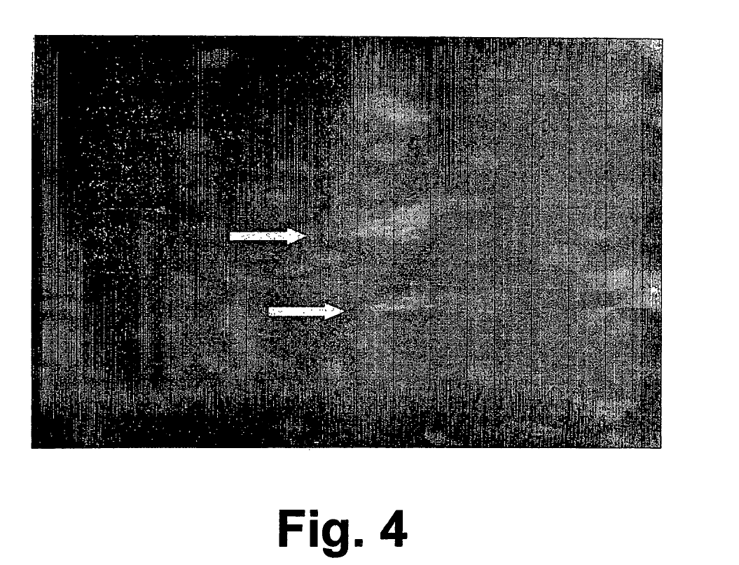

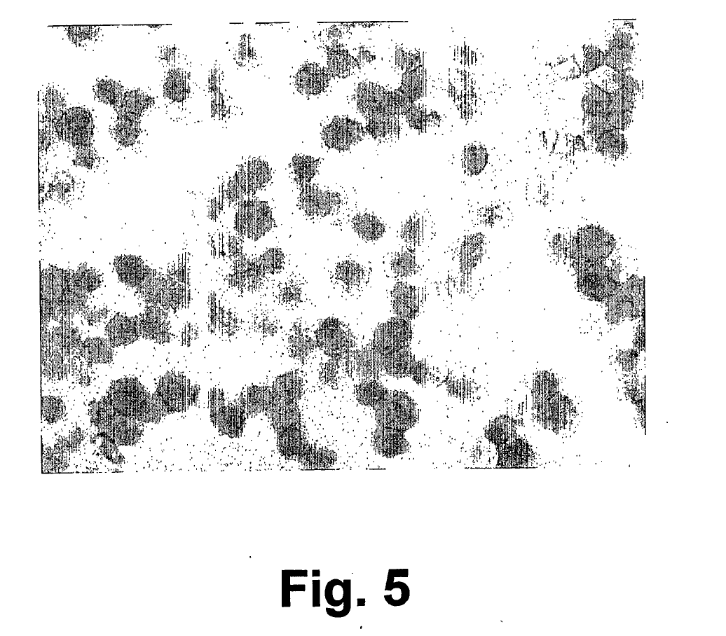

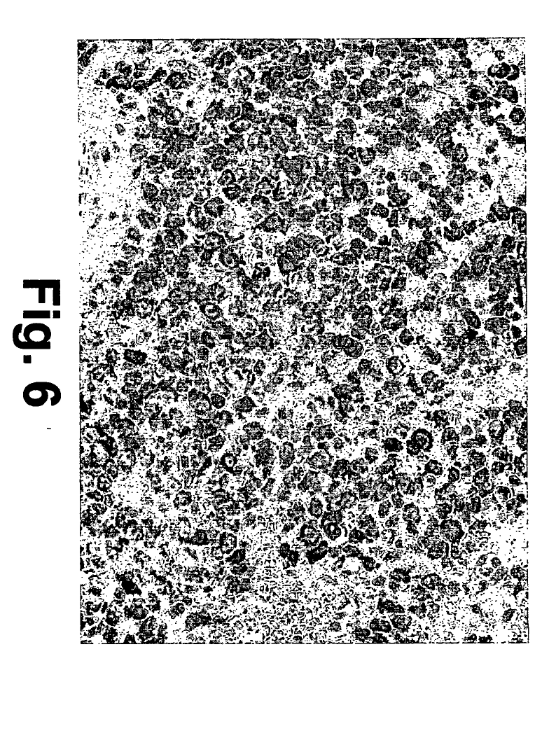

Homing of AC133+ cells in rat bone marrow—Human HLA-DR protein serves as a marker of endothelial cells and, as is further shown in FIG. 5, is expressed in human cord blood cells (dark brow stained cells). To evaluate the ability of transplanted stem cells to home into the bone marrow of recipient rats, bone marrow cells of AC133+ IV-injected rats were subjected to an HLA-DR immunohistochemistry analysis. As is shown by the intense dark brown staining in FIG. 6, the human transplanted stem cells home to the recipient bone marrow cells.

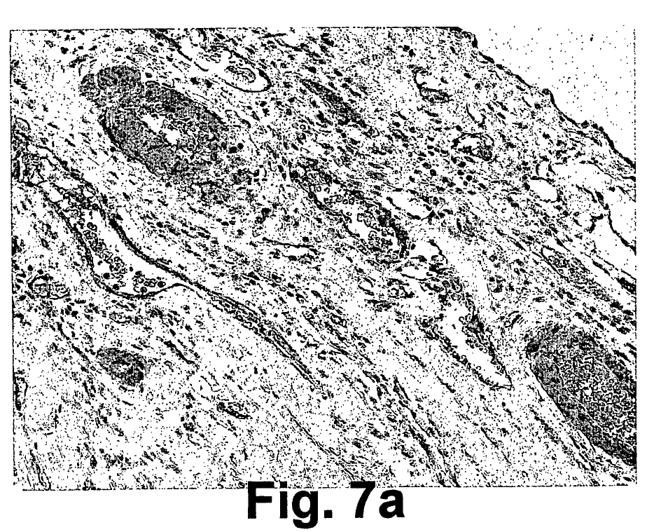

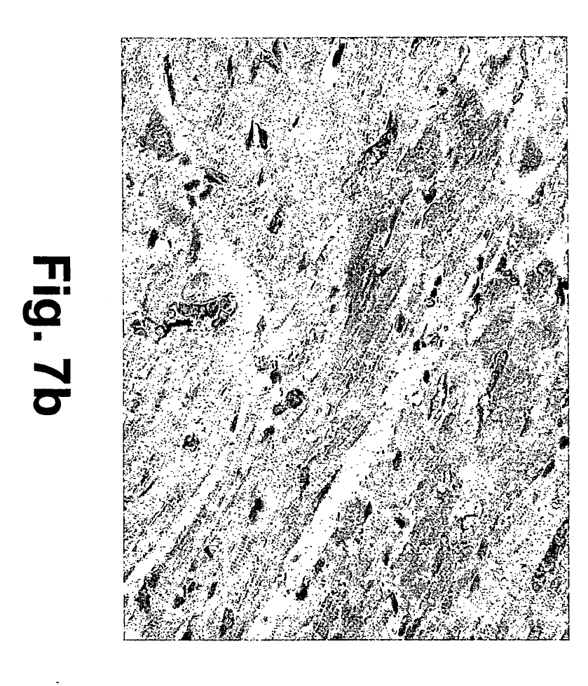

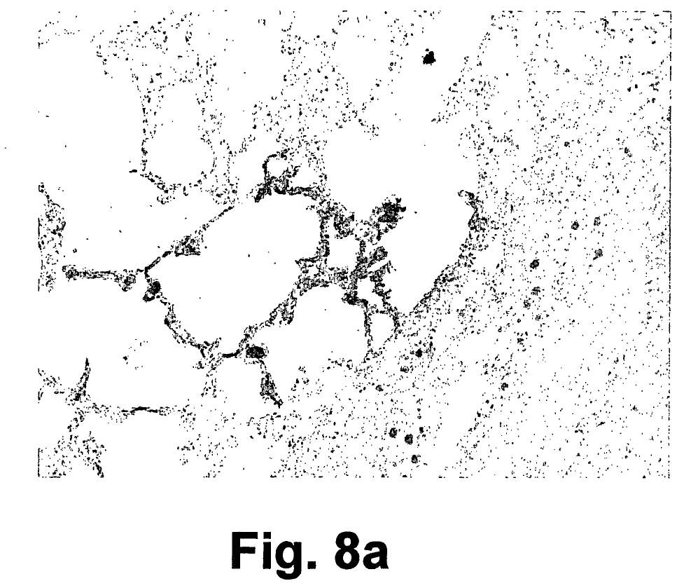

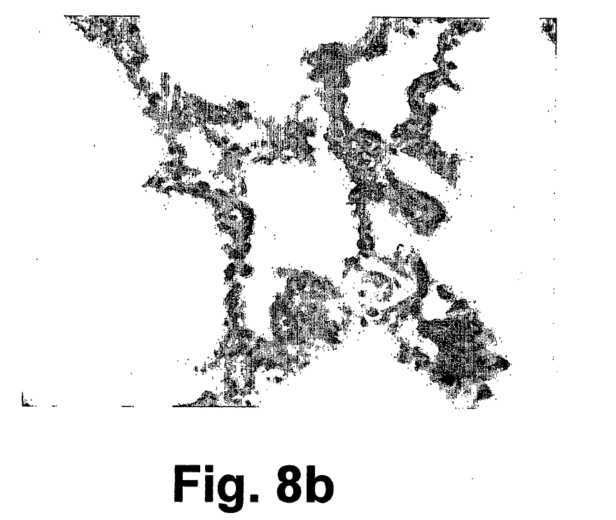

Trans-differentiated cells appeared in ischemic hearts and injured lung parenchyma—To further follow the homing capacity of transplanted stem cells, rat heart and lung sections were subjected to an HLA-DR immunohistochemistry analysis. HLA-DR—positive cells were observed around heart vessels (FIG. 7a, brown staining) and myocardium cells (FIG. 7b, brown staining) of AC133+ transplanted rats. In addition, in the process of causing the myocardial infarction, lung damage was sustained by one of the rats. In this rat, a positive HLA-DR staining was observed in the lung tissue (FIGS. 8a, b, brown staining). Thus, the AC133+ stem cells are capable of homing into ischemic heart and injured lung parenchyma.

Altogether, these findings demonstrate the capacity of umbilical cord blood stem cells, which are ex vivo expanded in the presence of copper chelators according to the teachings of the present invention, to trans-differentiate in vivo and to home into a variety of tissues in response to injury. Thus, copper chelator—mediated ex vivo—expanded stem cells are highly suitable for cell replacement and tissue regeneration therapies requiring cells with broad tissue homing and trans differentiation capacities.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-488. (canceled)

489. A method of in vivo differentiating stem cells into cells of a target tissue, the method comprising:
(a) obtaining a population of ex vivo expanded stem cells, said stem cells having been derived from a donor tissue; and
(b) administering said stem cells to the target tissue, so as to induce differentiation of the stem cells into at least one cell type characterizing the target tissue.

490. The method of claim 489, wherein said donor tissue has phenotypic and functional characteristics which are identical to those of the target tissue.

491. The method of claim 489, wherein said donor tissue has phenotypic and functional characteristics which are different from those of the target tissue.

492. The method of claim 489, wherein said stem cells derived from said donor tissue are selected from the group consisting of embryonic stem cells and neonatal and/or adult stem cells.

493. The method of claim 492, wherein said embryonic stem cells are selected from the group consisting of embryonic stem cells and embryonic germ cells.

494. The method of claim 492 wherein said neonatal and/or adult stem cells are selected from the group consisting of hematopoietic stem cells and nonhematopoietic stem cells.

495. The method of claim 494, wherein said hematopoietic stem cells are selected from the group consisting of bone marrow cells, neonatal umbilical cord blood cells and peripheral blood cells.

496. The method of claim 495, wherein said hematopoietic stem cells derived from said donor tissue are CD34+ enriched cells.

497. The method of claim 495, wherein said hematopoietic stem cells derived from said donor tissue are AC133+ enriched cells.

498. The method of claim 496, wherein said ex vivo expanded stem cells are characterized by downregulated expression of cell surface antigens CD38, CD3, CD61, CD19, CD33, CD14, CD15 and/or CD4.

499. The method of claim 496, wherein said non-hematopoietic stem cells are selected from the group consisting of neuronal stem cell, neuronal progenitor cells, oligodendrocyte progenitors, mesenchymal stem cells, hepatocyte stem cells, liver stem cells, epidermal stem cells, cardiac stem cells.

500. The method of claim 489, wherein said stem cells derived from the donor tissue are mixed with committed cells.

501. The method of claim 489, wherein said stem cells derived from the donor tissue are obtained from a donor which is syngeneic, allogeneic and/or xenogeneic with respect to a subject having said target tissue.

502. The method of claim 489, wherein said target tissue which comprises endodermal cells, ectodermal cells and/or mesodermal cells.

503. The method of claim 502, wherein said target tissue which comprises endodermal cells is selected from the group consisting of pharynx, esophagus, stomach, intestines, liver, pancreas, trachea and lungs.

504. The method of claim 502, wherein said target tissue which comprises ectodermal cells is selected from the group consisting of brain, adrenal gland, retina and epidermal skin.

505. The method of claim 502, wherein said target tissue which comprises mesodermal cells is selected from the group consisting of connective tissue, mesenchyme, bone, cartilage, muscle, fibrous tissue, dermal skin, heart, bone marrow and tubules of the urogenital system.

506. The method of claim 489, wherein said stem cells derived from the donor tissue are of an endodermal origin, an ectodermal origin and/or a mesodermal origin.

507. The method of claim 489, wherein said obtaining said population of ex vivo expanded stem cells is effected by culturing stem cells under conditions suitable for inducing cell proliferation and suppressing cell differentiation.

508. The method of claim 507, wherein said conditions suitable for inducing said cell proliferation and suppressing said cell differentiation are selected for reducing an expression and/or activity of CD38 in said stem cells.

509. The method of claim 507, wherein said conditions comprise nutrients and cytokines.

510. The method of claim 509, wherein said cytokines are early acting cytokines.

511. The method of claim 510, wherein said early acting cytokines are selected from the group comprising stem cell factor, FLT3 ligand, interleukin-1, interleukin-2, interleukin-3, interleukin-6, interleukin-10, interleukin-12, tumor necrosis factor-a and thrombopoietin.

512. The method of claim 509, wherein said cytokines are late acting cytokines.

513. The method of claim 512, wherein said late acting cytokines are selected from the group comprising granulocyte colony stimulating factor, granulocyte/macrophage colony stimulating factor, erythropoietin, FGF, EGF, NGF, VEGF, LIF, Hepatocyte growth factor and macrophage colony stimulating factor.

514. The method of claim 507, wherein said conditions comprise providing the cells with a transition metal chelator or chelate.

515. The method of claim 514, wherein said transition metal chelator or chelate is selected from the group consisting of polyamine chelating agents, ethylendiamine, diethylenetriamine, triethylenetetramine, triethylenediamine, tetraethylenepentamine, aminoetliylethanolamine, aminoethylpiperazine, pentaethylenehexamine, triethylenetetramine-hydrochloride, tetraethylenepentaminehydrochloride, pentaethylenehexamine-hydrochloride, tetraethylpentamine, captopril, penicillamine, N,N′-bis(3-aminopropyl)-1,3-propanediamine, N, N, Bis(2 animoethyl) 1,3 propane diamine, 1,7-dioxa-4,10-diazacyclododecane, 1,4,8,11-tetraaza cyclotetradecane-5,7-dione, 1,4,7-triazacyclononane trihydrochloride, 1-oxa-4,7,10triazacyclododecane, 1,4,8,12-tetraaza cyclopentadecane, 1,4,7,10-tetraaza cyclododecane.

516. The method of claim 507, wherein said conditions suitable for inducing said cell proliferation and suppressing said cell differentiation comprise culturing the cells with a PI 3-kinase activity or expression inhibitor.

517. The method of claim 516, wherein said PI 3-kinase expression inhibitor is a polynucleotide.

518. The method of claim 517, wherein said polynucleotide encodes a PI 3kinase intracellular antibody.

519. The method of claim 517, wherein said polynucleotide encodes a PI 3kinase antibody.

520. The method of claim 517 wherein said polynucleotide is a small interfering polynucleotide molecule directed to cause intracellular PI 3-kinase mRNA or gene degradation.

521. The method of claim 520, wherein said small interfering polynucleotide molecule is selected from the group consisting of an RNAi molecule, an anti-sense molecule, a ribozyme molecule and a DNAzyme molecule.

522. The method of claim 517, wherein said polynucleotide is a DNA vector containing a PI 3-kinase dominant negative construct.

523. The method of claim 516, wherein said PI 3-kinase activity inhibitor is selected from the group consisting of wortmannin and LY294002.

524. The method of claim 507, wherein said conditions suitable for inducing said cell proliferation and suppressing said cell differentiation are selected capable of reducing a capacity of said stem cells to respond to retinoids.

525. The method of claim 524, wherein reducing said capacity of said stem cells to respond to said retinoids is effected in a reversible manner.

526. The method of claim 524, wherein said conditions selected capable of reducing said capacity of said stem cells to respond to retinoids comprise the presence of an effective amount of at least one retinoic acid receptor antagonist, at least one retinoid X receptor antagonist and/or at least one Vitamin D receptor antagonist.

527. The method of claim 526, wherein said presence of said effective amount of at least one retinoic acid receptor antagonist, at least one retinoid X receptor antagonist and/or at least one Vitamin D receptor antagonist is effected for a time period of 0.1-50% of an entire ex vivo culturing period of said stem cells.

528. The method of claim 524, wherein said at least one retinoic acid receptor antagonist is selected from the group consisting of: AGN 194310; AGN 193109; 3-(4-Methoxy-phenylsulfanyl)-3-methyl-butyric acid; 6-Methoxy-2,2-dimethyl-thiochroman-4-one, 2,2-Dimethyl-4-oxo-thiochroman-6-yltrifluoromethane-sulfonate; Ethyl 4-((2,2 dimethyl-4-oxo-thiochroman-6-yl)ethynyl)-benzoate; Ethyl 4-((2,2-dimethyl-4-triflouromethanensulfonyloxy-(2H)-tluoclzromen-6-yl)ethynyl)-benzoate (41); Thiochromen-6-yl]-ethynyl]-benzoate (yl); (p-[(E)-2-[3′4′-Dihydro-4,4′-dimethyl-7′-(heptyloxy)-2′H-1-benzothiopyran-6′yl]propenyl]benzoic acid 1′1′-dioxide; 2E, 4E, 6E-[7-(3,5-Di-t-butyl-4-n-butoxyphenyl)-3-methyl]-octa-2,4,6-trienoic acid; 2E, 4E, 6E-[7-(3,5-Di-t-butyl-4-n-propoxyphenyl)-3-methyl]-octa-2,4,6-trienoic acid; 2E, 4E, 6E-[7-(3,5-Di-t-butyl-4-n-pentoxyphenyl)-3-methyl]-octa-2,4,6-trienoic acid; 2E, 4E, 6E-[7-(3,5-Di-t-butyl-4-n-hexoxyphenyl)-3-methyl]-octa-2,4,6-trienoic acid; 2E, 4E, 6E-[7-(3,5-Di-t-butyl-4-n-heptoxyphenyl)-3-methyl]-octa-2,4,6-trienoic acid; 2E, 4E, 6E-[7-(3,5-Di-t-butyl-4-n-octoxyphenyl)-3-methyl]-octa-2,4,6-trienoic acid; (2E, 4E, 6E)-7-[3-t-butyl-5(1-phenyl-vinyl)-phenyl]-3-methyl-octa-2,4,6-trienoic acid; 2E, 4E, 6E-[7(3,5-Di-t-butyl-4-{[4,5-H2]-n-pentoxy}phenyl)-3-methyl]-octa-2,4,6-trienoic acid; (2E, 4E)-(IRS, 2RS)-5-[2-(3,5-di-tert.butyl-2-ethoxy-phenyl)cyclopropyl]-3-methyl-penta-2,4-dienoic acid ethyl ester; (2E, 4E)-(1RS, 2RS)-5-[2-(3,5-di-tert.butyl-2-ethoxy-phenyl)-cyclopropyl]-3-methyl-penta-2,4-dienoic acid; (2E, 4E)-(1RS, 2RS)-5-[2-(3,5-di-tert.butyl-2-butoxy-phenyl)-cyclopropyl]-3-methyl-penta-2,4-dienoic acid; (2E, 4E, 6Z)-7-[3,5-di-tert.butyl-2-ethoxyphenyl]3-methyl-2,4,6-octatrienoic acid; (2E, 4E, 6Z)-7-[3,5-di-tert.butyl-2-butyloxyphenyl]-3-methyl-2,4,6-octatrienoic acid; 4-(5,6,7,8-tetrahydro-5,5,8,8-tetramethyl-2-naphthalene-carboxamido) benzoic acid; (2E, 4E)-3-methyl-5-[(1S, 2S)-2-(5,5,8,8-tetramethyl-5,6,7,8-tetrahydro-naphthalen-2-yl)-cyclopropyl]-penta-2,4-dienoic acid; p-[(E)-2-[3′,4′-Dihydro-4′,4′-dimethyl-7′-(heptyloxy)-2′H-1-benzothiopyran-6′-yl]propenyl]benzoic acid; 1′,1′-dioxide, 4-(7,7,10,10-Tetramethyl-1-pyridin-3-ylmethyl-4,5,7,8,9,10-hexahydro-1H-naphto [2,3-g]indol-3-yl)-benzoic acid; (2E, 4E, 6Z)-7-[3,5-di-tert.butyl-2-methoxyphenyl]-3-methyl-2,4,6-octatrienoic acid; (2E, 4E, 6Z)-7-[3,5-di-tert.butyl-2-ethoxyphenyl]-3-methyl-2,4,6-octatrienoic acid; (2E, 4E, 6Z)-7-[3,5-di-tert.butyl-2-hexyloxyphenyl]-3-methyl-2,4,6-octatrienoic acid; (2E, 4E, 6Z)-7-[3,5-di-tert.butyl-2-octyloxyphenyl]-3-methyl-2,4,6-octatrienoic acid; and (2E, 4E)-(1RS, 2RS)-5-[2-(3,5-di-tert-butyl-2-butoxy-phenyl)-cyclopropyl]-3-methyl-penta-2,4-dienoic acid, (2E, 4E, 6Z)-7-(3-n-propoxy-5,6,7,8-tetrahydro-5,5,8,8-tetramethylnaphthalene-2-yl)-3-methylocta-2,4,6-trienoic acid, and 4-(5H-2,3(2,5 dimethyl-2,5-hexano)-5-n-propyldibenzo [b,e][1,4]diazepin-11-yl)benzoic acid, 4-(5H-2,3-(2,5-dimethyl-2,5-hexano)-5methyl-8-nitrodibenzo[b,e][1,4]diazepin-11-yl)benzoic acid, 4-{[4-(4-Ethylphenyl) 2,2-dimethyl-(2H)-thiochromen-6-yl]ethynyl}benzoic acid, 4-[4-2methyl-1,2-dicarba-closo-dodecaboran-1-yl-phenylcarbamoyl]benzoic acid, 4-[4,5,7,8,9,10-hexahydro-7,7,10,10-tetramethyl-1-(3-pyridylmethyl)-anthra[1,2-b]pyrrol-3-yl]benzoic acid, (3-pyridylmethyl)-]5-thiaanthra[2,1-b]pyrrol-3-yl)benzoic acid, and (3-pyridylmethyl)-anthra[2 ml-d]pyrazol-3-yl]benzoic acid.

529. The method of claim 526, wherein said at least one retinoid X receptor antagonist is selected from the group consisting of: LGN100572, LGN100574, 1-(3-hydroxy-5,6,7,8-tetrahydro-5,5,8,8-tetramethylnaphthalene-2-yl)ethanone, 1-(3-propoxy-5,6,7,8-tetrahydro-5,5,8,8-tetramethylnaphthalene-2-yl)ethanone, 3-(3-propoxy-5,6,7,8-tetrahydro-5,5,8,8-tetramethylnaphthalene-2-yl)but-2-enenitrile, 3-(3-propoxy-5,6,7,8-tetrahydro-5,5,8,8-tetramethylnaphthalene-2-yl)but-2-enal, (2E, 4E, 6E)-7-3 [-propoxy-5,6,7,8-tetrahydro 5,5,8,8-tetramethyl-2-naphthalene-2-yl]-3-methylocta-2,4,6-trienoic acid, 4-[3,5,5,8,8-pentamethyl-5,6,7,8-tetrahydro-2naphthyl)carbonyl]benzoic acid, 4-[1-(3,5,5,8,8-pentamethyl-5,6,7,8-tetrahydro-2naphthyl)ethenyl]benzoic acid, 4-[1 (3,5,5,8,8-pentamethyl-5,6,7,8-tetrahydro-2naphthyl)cyclopropyl]benzoic acid, 4-[1-(3,5,5,8,8-pentamethyl-5,6,7,8-tetrahydro-2-naphthyl)ethenyl]benzenete trazole, 2-[1-(5,5,8,8-tetramethyl-5,6,7,8-tetrahydro-2naphthyl) ethenyl]pyridine-5-carboxylic acid, 2-[1-(3,5,5,8,8-pentamethyl-5,6,7,8tetrahydro-2-naphthyl)ethyl]pyridine-5-carboxylic acid, ethyl-2-[1-(3,5,5,8,8pentamethyl-5,6,7,8-tetrahydro-2-naphthyl)ethenyl]pyridine-5-carboxylate, 5-[1-3,5,5,8,8-pentamethyl-5,6,7,8-tetrahydro-2-naphthyl)ethenyl]pyridine-2-carboxylic acid, 2-[1-(3,5,5,8,8-pentamethyl-5,6,7,8-tetrahydro-2-naphthyl)cyclopropyl]pyridine-5-carboxylic acid, methyl 2-[1-(3,5,5,8,8-pentamethyl-5,6,7,8tetrahydro-2-naphthyl)cyclopropyl]pyridine-5-carboxylate, 4-[1-(3,5,5,8,8pentamethyl-5,6,7,8-tetrahydro-2-naphtliyl)ethenyl]-N-(4-hydroxyplienyl)benzamide, 2-[1-(3,5,5,8,8-Pentamethyl-5,6,7,8-tetrallydro-2-napllthyl)ethenyl]pyridine-5carboxylic acid, 2-[1-(3,5,5,8,8-Pentamethyl-5,6,7,8-tetrahydro-2naphthyl)cyclopropyl]pyridine-5-carboxylic acid, 4-[(3,5,5,8,8-pentamethyl-5,6,7,8tetrahydro-2-naphthyl)carbonyl]benzoic acid butyloxime, 4-[(3,5,5,8,8-pentamethyl-5,6,7,8-tetrahydro-2-naphthyl)carbonyl]benzoic acid propyloxime, 4-[(3,5,5,8,8pentamethyl-5,6,7,8-tenahydro-2-naphthyl)carbonyl]benzoic acid cyanoimine, 4-[(3,5,5,8,8-pentamethyl-5,6,7,8-tetrahydro-2-naphthyl)carbonyl]benzoic acid allyloxime, 4-[(3,5,5,8,8-pentamethyl-5,6,7,8-tetrahydro-2-naphthyl)carbonyl]benzoic acid 4-(3-methylbut-2-enoic acid)oxime, and 4-[(3,5,5,8,8-pentamethyl-5,6,7,8-tetraliydro-2-naphthyl)carbonyl]benzoic acid 1-aminoethyloxime, (2E, 4E, 6Z)-7-(3-n-propoxy-5,6,7,8-tetrahydro-5,5,8,8-tetramethylnaphthalene-2-yl)-3-methylocta-2,4,6trienoic acid, and 4-(5H-2,3(2,5 dimethyl-2,5-hexano)-5-n-propyldibenzo[b,e][1,4]diazepin-11-yl)benzoic acid, and 4-(5H-2,3-(2,5-dimethyl-2,5-hexano)-5methyl-8-nitrodibenzo[b,e][1,4]diazepin-11-yl) benzoic acid.

530. The method of claim 526, wherein said at least one Vitamin D receptor antagonist is selected from the group consisting of: 1 alpha, 25-(OH)-D3-26, 23 lactone; 1alpha, 25-dihydroxyvitamin D (3); the 25-carboxylic ester ZK159222; (23S)-25-dehydro-1 alpha-OH-D (3); (23R)-25-dehydro-1 alpha-OH-D (3); 1 beta, 25 (OH) 2 D3; 1 beta, 25 (OH) 2-3-epi-D3; (23S) 25-dehydro-1 alpha (OH) D3-26, 231actone; (23R) 25-dehydro-1 alpha (OH) D3-26,23-lactone and Butyl-(5Z, 7E, 22E-(1S, 7E, 22E-(1S, 3R, 24R)-1,3,24-trihydroxy-26,27-cyclo-9,10-secocholesta-5,7,10(19), 22-tetraene-25-carboxylate).

531. The method of claim 524, wherein said conditions selected capable of reducing said capacity of said stem cells to respond to retinoids comprise a polynucleotide which downregulates retinoic acid receptor, retinoid X receptor and/or Vitamin D receptor.

532. The method of claim 531, wherein said polynucleotide encodes an anti retinoic acid receptor, an anti retinoid X receptor and/or an anti Vitamin D receptor intracellular antibody.

533. The method of claim 531, wherein said polynucleotide encodes an anti retinoic acid receptor, an anti retinoid X receptor and/or an anti Vitamin D receptor antibody.

534. The method of claim 531, wherein said polynucleotide is a small interfering polynucleotide molecule directed to cause intracellular retinoic acid receptor, retinoid X receptor or Vitamin D receptor mRNA or gene degradation.

535. The method of claim 534, wherein said small interfering polynucleotide molecule is selected from the group consisting of an RNAi molecule, an anti-sense molecule, a ribozyme molecule and a DNAzyme molecule.

536. The method of claim 524, wherein said conditions selected capable of reducing said capacity of said stem cells to respond to retinoids comprise an effective amount of at least one PI 3-kinase expression or activity inhibitor.

537. The method of claim 507, wherein said conditions suitable for inducing said cell proliferation and suppressing said cell differentiation are selected capable of reducing a capacity of said stem cells to respond to Vitamin D.

538. The method of claim 537, wherein reducing said capacity of said stem cells to respond to said Vitamin D is effected in a reversible manner.

539. The method of claim 537, wherein said conditions selected capable of reducing said capacity of said stem cells to respond to Vitamin D comprise the presence of an effective amount of at least one retinoic acid receptor antagonist, at least one retinoid X receptor antagonist and/or at least one Vitamin D receptor antagonist.

540. The method of claim 539, wherein said presence of said effective amount of at least one retinoic acid receptor antagonist, at least one retinoid X receptor antagonist and/or at least one Vitamin D receptor antagonist is effected for a time period of 0.1-50% of an entire ex vivo culturing period of the stem cells.

541. The method of claim 537, wherein said conditions selected capable of reducing said capacity of said stem cells to respond to Vitamin D comprise a polynucleotide which downregulates retinoic acid receptor, retinoid X receptor and/or Vitamin D receptor.

542. The method of claim 507, wherein said conditions suitable for inducing said cell proliferation and suppressing said cell differentiation are selected capable of reducing a capacity of said stem cells to respond to signaling pathways involving retinoic acid receptor.

543. The method of claim 542, wherein reducing said capacity of said stem cells to respond to signaling pathways involving retinoic acid receptor is effected in a reversible manner.

544. The method of claim 507, wherein said conditions suitable for inducing said cell proliferation and suppressing said cell differentiation are selected capable of reducing a capacity of said stem cells to respond to signaling pathways involving retinoid X receptor.

545. The method of claim 544, wherein reducing said capacity of said stem cells to respond to said signaling pathways involving retinoid X receptor is effected in reversible manner.

546. The method of claim 507, wherein said conditions suitable for inducing said cell proliferation and suppressing said cell differentiation are selected capable of reducing a capacity of said stem cells to respond to signaling pathways involving Vitamin D receptor.

547. The method of claim 546, wherein reducing said capacity of said stem cells to respond to signaling pathways involving Vitamin D receptor is effected in reversible manner.

548. A method of treating an individual suffering from a disorder requiring cell or tissue replacement comprising:
(a) subjecting isolated stem cells to culturing conditions selected suitable for inducing cell proliferation and suppressing cell differentiation, thereby obtaining an expanded stem cell population; and
(b) introducing said expanded stem cell population into a tissue of the individual associated with the disorder thereby inducing differentiation of cells of said expanded stem cell population into cells characterizing said tissue, thereby treating the individual suffering from the disorder requiring cell or tissue replacement.

549. The method of claim 548, wherein said conditions suitable for inducing said cell proliferation and suppressing said cell differentiation comprise providing the cells with a transition metal chelate or chelator.

550. The method of claim 549, wherein said transition metal chelate or chelator is selected from the group consisting of polyamine chelating agents, ethylendiamine, diethylenetriamine, triethylenetetramine, triethylenediamine, tetraethylenepentamine, aminoethylethanolamine, aminoethylpiperazine, pentaethylenehexamine, triethylenetetramine-hydrochloride, tetraethylenepentaminehydrochloride, pentaethylenehexamine-hydrochloride, tetraethylpentamine, captopril, penicilamine, N,N′-bis(3-aminopropyl)-1,3-propanediamine, N, N, Bis (2 animoethyl) 1,3 propane diamine, 1,7-dioxa-4,10-diazacyclododecane, 1,4,8,11-tetraaza cyclotetradecane-5,7-dione, 1,4,7-triazacyclononane trihydrochloride, 1-oxa-4,7,10triazacyclododecane, 1,4,8,12-tetraaza cyclopentadecane, 1,4,7,10-tetraaza cyclododecane.

551. The method of claim 548, wherein said isolated stem cells are hematopoietic stem cells.

552. The method of claim 551, wherein said hematopoietic stem cells are selected from the group consisting of bone marrow cells, neonatal umbilical cord blood cells and peripheral blood cells.

553. The method of claim 551, wherein said hematopoietic stem cells are CD34+ enriched cells.

554. The method of claim 551, wherein said hematopoietic stem are AC133+ enriched cells.

555. The method of claim 548, wherein said expanded stem cell population is characterized by downregulated expression of cell surface antigens CD38, CD3, CD61, CD19, CD33, CD14, CD15 and/or CD4.

556. The method of claim 548, wherein said isolated stem cells are mixed with committed cells.

557. The method of claim 548, wherein said isolated stem cells are obtained from a donor which is syngeneic, allogeneic and/or xenogeneic with respect to said tissue of said individual associated with said disorder.

558. The method of claim 548, wherein said tissue of said individual associated with the disorder comprises endodermal cells, ectodermal cells and/or mesodermal cells.

559. The method of claim 558, wherein said tissue comprises endodermal cells is selected from the group consisting of pharynx, esophagus, stomach, intestines, liver, pancreas, trachea and lungs.

560. The method of claim 558, wherein said target tissue which comprises ectodermal cells is selected from the group consisting of brain, adrenal gland, retina and epidermal skin.

561. The method of claim 558, wherein said target tissue which comprises mesodermal cells is selected from the group consisting of connective tissue, mesenchyme, bone, cartilage, muscle, fibrous tissue, dermal skin, heart, bone marrow and tubules of the urogenital system.

562. The method of claim 558, wherein said disorder is selected from the group consisting of a neurological disorder, a muscular disorder, a cardiovascular disorder, an hematological disorder, a skin disorder, a liver disorder, and a pancreas disorder.

563. A method of in-tissue differentiating adult stem cells into cells of a predetermined type comprising:
(a) culturing the adult stem cells obtained from a donor tissue under conditions selected suitable for inducing cell proliferation and suppressing cell differentiation, thereby obtaining an expanded stem cell population; and
(b) introducing said expanded adult stem cell population into a target tissue of a predetermined type to thereby differentiate said expanded stem cell population into cells characterizing said target tissue.

564. The method of claim 564, wherein said donor tissue has phenotypic and functional characteristics which are identical to those of said target tissue.

565. The method of claim 563, wherein said donor tissue has phenotypic and functional characteristics which are different from those of said target tissue.

566. The method of claim 563, wherein the adult stem cells obtained from said donor tissue are syngeneic, allogeneic and/or xenogeneic with respect to said cells characterizing said target tissue.

567. A method of in-tissue differentiating bone marrow derived stem cells into cells of a predetermined type comprising:
(a) culturing the bone marrow cells obtained from a donor tissue under conditions selected suitable for inducing cell proliferation and suppressing cell differentiation, thereby obtaining an expanded stem cell population; and
(b) introducing said expanded bone marrow derived stem cell population into a target tissue of a predetermined type to thereby differentiate said expanded stem cell population into cells characterizing said target tissue.

568. The method of claim 567, wherein said bone marrow derived stem cells are mixed with committed cells.

569. The method of claim 567, wherein said bone marrow derived stem cells are obtained from a donor which is syngeneic, allogeneic and/or xenogeneic with respect to said target tissue associated with said disorder.

570. The method of claim 567, wherein said target tissue associated with the disorder comprises endodermal cells, ectodermal cells and/or mesodermal cells.

571. The method of claim 570, wherein said tissue comprises endodermal cells is selected from the group consisting of pharynx, esophagus, stomach, intestines, liver, pancreas, trachea and lungs.

572. The method of claim 570, wherein said target tissue which comprises ectodermal cells is selected from the group consisting of brain, adrenal gland, retina and epidermal skin.

573. The method of claim 570, wherein said target tissue which comprises mesodermal cells is selected from the group consisting of connective tissue, mesenchyme, bone, cartilage, muscle, fibrous tissue, dermal skin, heart, bone marrow and tubules of the urogenital system.

574. The method of claim 568, wherein the target tissue is cardiac tissue.

575. The method of claim 574, wherein the cardiac tissue is post myocardial infarct.
